(19) United States
(12) Patent Application Publication
(10) Pub. No.: US 2026/0209390 A1
(43) Pub. Date: Jul. 23, 2026

(54) METHOD FOR GENERATING BISPECIFIC PROTEINS

(71) Applicant: MERUS N.V., Utrecht (NL)

(72) Inventors: Cornelis Adriaan DE KRUIF, Utrecht (NL); Franziska MORTENSEN, Utrecht (NL)

(21) Appl. No.: 19/142,758

(22) PCT Filed: Dec. 22, 2023
(86) PCT No.: PCT/NL2023/050681

§ 371 Date: Jun. 24, 2025

(30) Foreign Application Priority Data
Dec. 27, 2022 (NL) 2033843

(51) Int. Cl. C07K 16/46

(52) U.S. Cl. CPC C07K 16/468; C07K 2317/14; C07K 2317/21; C07K 2317/31; C07K 2317/524; C07K 2317/526; C07K 2317/732

(57) ABSTRACT

Provided herein is a method for producing a heterodimeric protein that comprises two distinct IgG CH3 domains that are capable of forming a CH3-CH3 interface. Further, provided herein is an isolated heterodimeric protein obtained by said method. Provided herein is also an isolated heterodimeric antibody comprising a first IgG CH3 domain and a second IgG CH3 domain, wherein the first CH3 domain and the second CH3 domain are capable of forming a CH3-CH3 interface, as well as a pharmaceutical composition comprising said isolated heterodimeric proteins.

### FIELD OF INVENTION

Provided herein is a method for producing a heterodimeric protein that comprises two distinct IgG CH3 domains that are capable of forming a CH3-CH3 interface. Further, provided herein is an isolated heterodimeric protein obtained by said method. Provided herein is also an isolated heterodimeric antibody comprising a first IgG CH3 domain and a second IgG CH3 domain, wherein the first CH3 domain and the second CH3 domain are capable of forming a CH3-CH3 interface capable of generating multispecific binding domains, as well as a pharmaceutical composition comprising said isolated heterodimeric proteins having multiple binding specificities.

### BACKGROUND

Monospecific antibodies play an important role as therapeutic molecules of many diseases, in particular for the treatment of cancer. Monospecific antibodies bind to a single specific area, or epitope, of an antigen and, for use in therapy, are often selected for a desirable functional property (such as for example killing of tumour cells, blocking of receptor-ligand interactions or virus neutralization). Monospecific antibodies have many beneficial characteristics, such as they can be produced at large quantities, and their biophysical and biochemical characteristics can be analysed in great detail to ensure batch-to-batch consistency, which facilitates regulatory acceptability.

Despite these favourable characteristics, monospecific antibodies have several disadvantages associated with their specificity. For this reason, in the recent years, bispecific and multispecific antibodies have started to play an even more important role as they have the potential to overcome some of the limitations of monospecific antibody therapy. For example, they can be used as mediators to target a drug or toxic compound to target cells, as mediators to retarget effector mechanisms to disease-associated sites or as mediators to increase specificity for tumour cells, for example by binding to a combination of target molecules that is exclusively found on tumour cells.

While desirable, the manufacture and testing of multispecific antibodies still poses challenges. For example, bispecific antibodies based on the IgG format, consisting of two heavy chains and two light chains have been produced by a variety of methods. For instance, bispecific antibodies may be produced by fusing two antibody-secreting cell lines to create a new cell line or by expressing two antibodies in a single cell using recombinant DNA technology. These approaches yield multiple antibody species as the respective heavy chains from each antibody may form monospecific dimers (also called homodimers or homodimeric antibodies), which contain two identical paired heavy chains with the same specificity, and bispecific dimers (also called heterodimers or heterodimeric antibodies) which contain two different paired heavy chains with different specificity. In addition, light chains and heavy chains from each antibody may randomly pair to form inappropriate, non-functional combinations. This problem is known as heavy and light chain mispairings. This problem can be solved by choosing antibodies that share a common light chain for expression as a bispecific. When a common light chain is used, expression of two heavy chains and one common light chain in a single cell may result in 3 different antibody species (i.e. two monospecific ‘parental’ antibodies and the bispecific antibody) so that the bispecific antibody of interest needs to be purified from the resulting antibody mixture.

Whereas a single cell may produce essentially a single antibody species through use of heterodimerization technology that pairs the constant region of the bispecific, to achieve this product requires use technology that limits the ability to combine different binding domains (e.g., a common light chain binding domain with a non-common light chain antibody). Thus, there remains a need for multispecific formats that permit the combination of different binding domains (common and non-common light chains) to generate essentially a single product, which can be tested preclinically, and manufactured reliably for clinical and commercial development.

### SUMMARY OF INVENTION

The present disclosure is based on the inventors development of a novel method for producing heterodimeric proteins, in particular heterodimeric antibodies, which employs a novel Fab-arm exchange method.

The method described herein is based on the inventors' development of a novel Fab-arm exchange method. This method involves introducing variations in the CH3 domain, which permit Fab-arm exchange to occur among all IgG subtypes without the need to destabilise the core hinge region. As discussed in more detail in the examples section below, the inventors have shown that Fab-arm exchange in an IgG1 molecule may occur when one of the CH3 domains comprises amino acids 351D and 368E and the other CH3 domain comprises amino acids 366K and 351K. Whilst the examples employ these specific amino acids at positions 351, 366 and 368, the method will likewise work when a different positively charged amino acid residue at position 351 and 366 is present on one CH3 domain, and a corresponding negatively charged amino acid residue at position 351 and 368 is present on the other CH3 domain.

One advantage of the present disclosure is that it permits the exchange of Fab domains with any previously disclosed antibody for which its amino acid sequence is given, by introducing in its CH3 domain positively charged amino acids at position 351 and 366 and exchange its Fab domain with a second antibody that contains in its CH3 domain, negatively charged amino acids at positions 351 and 368. Currently, more than 800 antibodies are known from the International Nonproprietary Names (INN) list as kept by the World Health Organization, either being approved or in development, which can all be combined with another antibody, as long as both antibodies comprise two distinct IgG CH3 domains that are capable of forming a CH3-CH3 interface, wherein one of the antibodies has a CH3 domain with positively charged amino acids at position 351 and 366 and the other antibody has a CH3 domain with negatively charged amino acids at positions 351 and 368.

Generating a multispecific antibody comprising binding domains not sharing an identical light chain, the expression of such a product from a single cell will create light chain mispairing, resulting in numerous different antibody species with lost or reduced affinity or specificity that require laborious, time consuming and inefficient means of separation to select the desired species. The present disclosure now permits antibodies with common light chain binding domains to be combined with antibodies with non-common light chain binding domains to generate multispecific antibody species at relative purity, yield and efficiency. Also, the present disclosure now permits antibodies with non-common light chain binding domains to be combined with other antibodies with non-common light chain binding. The light chains can be any members from both the kappa and lambda families. The Fab domains can be from any source including from common light chain repertoires.

The present disclosure, in addition to allowing for Fab-arm exchange to be utilised with all IgG formats, is also associated with certain unexpected advantages. For example, it has now been found that upon exposure to reducing and reoxidising conditions half-antibodies comprising amino acid combinations 366K and 351K, or 351D and 368E, remain predominantly as half-antibodies, rather than to bind other half-antibodies with the same residues. As it will be clear to a person with skill in the art, this is advantageous in the context of producing bispecific antibodies, as the tendency of half-antibodies to form homodimers may hinder the efficient generation and purification of bispecific antibodies. Also, the presence of homodimers hampers screening large repertoires of multispecific antibodies for functional activities. As shown in more detail in the Examples section of the present application, compared to a Fab arm exchange method known in the art, which relies on using 405L/409R variations, the novel method described herein may be much better at reducing the production of potentially undesired homodimers. Specifically, as described in Example 7 of the present disclosure, the method of the present invention resulted in the production of only about 1% homodimers, vs. 5 to 13% obtained when using the 405L/409R method. By the same token, the method of the invention may produce a higher amount of heterodimers, as also shown in Example 7.

Accordingly, in a first aspect, provided herein is a method for producing a heterodimeric protein that comprises two distinct IgG CH3 domains that are capable of forming a CH3-CH3 interface, said method comprising the steps of:
providing:
   (a) a first protein comprising a first CH3 domain which comprises a positively charged amino acid residue at position 351 and 366, and
   (b) a second protein comprising a second CH3 domain which comprises a negatively charged amino acid residue at position 351 and 368,
   wherein the numbering is according to EU numbering,
incubating the proteins of (a) and (b) together under reducing conditions to provide a reduced first protein and a reduced second protein; and
reoxidising the reduced proteins to obtain the heterodimeric protein.

In certain aspects, the first protein and/or the second protein comprises or is selected from the group consisting of: a monomeric protein, a homodimeric protein and a heterodimeric protein.

In certain aspects, the IgG CH3 domain is an IgG1, an IgG2, an IgG3, or an IgG4 CH3 domain.

In certain aspects, the IgG CH3 domain is an IgG1 CH3 domain.

In certain aspects, the IgG CH3 domain of the first protein and of the second protein are an IgG1.

In certain aspects, the IgG CH3 domain of the first protein and of the second protein are an IgG2.

In certain aspects, the IgG CH3 domain of the first protein and of the second protein are an IgG3.

In certain aspects, the IgG CH3 domain of the first protein and of the second protein are an IgG4.

In certain aspects, the IgG CH3 domain is a human IgG CH3.

In certain aspects, the first protein and the second protein comprise an identical hinge region. In certain aspects, the first protein and the second protein comprise an IgG1 hinge region. In certain aspects, the first protein and the second protein comprise an IgG2 hinge region. In certain aspects, the first protein and the second protein comprise an IgG3 hinge region. In certain aspects, the first protein and the second protein comprise an IgG4 hinge region.

In certain aspects, the first protein and/or the second protein comprises or is selected from the group consisting of: an antibody and a half-antibody, or a fragment thereof.

In certain aspects the antibody, half-antibody, or fragment thereof is a human antibody, half-antibody, or fragment thereof.

In certain aspects, the fragment is a monomeric Fc region or a dimeric Fc region.

In certain aspects, the antibody, half antibody, or fragment thereof is an IgG1, an IgG2, an IgG3, or an IgG4 antibody, half antibody, or fragment thereof. In certain aspects, the first protein is an antibody comprising a first binding specificity and the second protein may be an antibody comprising a second distinct binding specificity.

In certain aspects, the first protein is an antibody comprising a binding specificity and the second protein may be an antibody comprising another distinct binding specificity.

In certain aspects, the first protein is a multispecific antibody (for instance a bispecific or trispecific antibody). In certain aspects, the second protein is a multispecific antibody (for instance a bispecific or trispecific antibody). In certain aspects, both the first and second proteins are a multispecific antibody (for instance a bispecific or trispecific antibody).

In certain aspects, the first protein and/or the second protein is a homodimeric antibody.

In certain aspects, the first protein and/or the second protein is a heterodimeric antibody comprising a common light chain.

In certain aspects, the heterodimeric protein as obtained is a heterodimeric antibody.

In certain aspects, the heterodimeric antibody is multivalent, optionally wherein the multivalent antibody comprises two or more valences, including a bivalent, trivalent or tetravalent antibody.

The present disclosure is suitable for the generation of multivalent multimers known in the art, which comprise and do not comprise use of a common light chain. WO2019/190327 is incorporated by reference and FIG. 1a-1u thereof in particular.

In certain aspects, the heterodimeric antibody is a multispecific antibody, optionally wherein the multispecific antibody comprises two or more valences and is a bispecific, trispecific, tetraspecific antibody, or having up to six valences.

In certain aspects, the heterodimeric antibody may comprise two non-identical light chains.

In certain aspects, the first CH3 domain may comprise: 351K and 366R, 351R and 366K, 351K and 366K, or 351R and 366R.

In certain aspects, the second CH3 domain may comprise: 351D and 368E, 351E and 368D, 351D and 368D, or 351E and 368E.

In certain aspects, the second CH3 domain may comprise 351D and 368E and the first CH3 domain may comprise 366K and 351K.

In certain aspects, the second protein is obtained independently from the first protein.

In certain aspects, the reducing conditions may comprise:
(a) incubating the proteins in the presence of any suitable reducing agent known in the art, optionally wherein the reducing agent comprises or is selected from the group consisting of: 2-mercaptoethylamine (2-MEA), dithiothreitol (DTT), dithioerythritol (DTE), glutathione, tris(2-carboxyethyl)phosphine (TCEP), L-cysteine, beta-mercapto-ethanol, thioglycolate, cysteamine, homocysteine, penicillamine, and sodium borohydride; and/or
(b) incubating the proteins at a pH of between 6.0 and 12.0, optionally wherein the pH is between 7.0 and 11.0; and/or
(c) incubating the proteins at a redox potential of between −150 and −600 mV, optionally wherein the redox potential is between −250 and −400 mV.

In certain aspects, the method may further comprise the step of enriching for and/or isolating the heterodimeric protein as obtained after reoxidisation.

In certain aspects, the heterodimeric protein obtained after reoxidisation is enriched for and/or isolated using a method that comprises or is selected from the group consisting of: precipitation, centrifugation, filtration, size-exclusion chromatography, affinity chromatography, cation- and/or anion-exchange chromatography, and hydrophobic interaction chromatography.

The method described herein provides a method for producing a heterodimeric antibody that comprises two distinct IgG CH3 domains that are capable of forming a CH3-CH3 interface, said method comprising the steps of:
providing:
   (a) a first IgG antibody or half-body, wherein each CH3 domain comprises a positively charged amino acid residue at position 351 and 366, and
   (b) a second IgG antibody or half body, wherein each CH3 domain comprises a negatively charged amino acid residue at position 351 and 368,
   wherein the numbering is according to EU numbering,
incubating the antibodies and/or half-bodies of (a) and (b) together under conditions sufficient to allow cysteines in the core hinge regions of the antibodies or half-bodies to undergo disulphide-bond isomerization to obtain the heterodimeric protein.

In a further aspect, provided herein is an isolated heterodimeric protein obtainable by the methods of the disclosure.

In certain aspects, the heterodimeric protein obtainable by the methods of the disclosure is an IgG antibody.

In certain aspects, the heterodimeric protein obtainable by the methods of the disclosure is a multispecific IgG antibody.

In certain aspects, the IgG antibody comprises two light chains that have non-identical sequences. In certain aspects, the IgG antibody comprises binding domains comprising non-identical light chain sequences.

An isolated heterodimeric antibody comprising a first IgG CH3 domain and a second IgG CH3 domain, wherein the first CH3 domain and the second CH3 domain are capable of forming a CH3-CH3 interface, wherein the first CH3 domain comprises one or more of the amino acid variants 366K, 366R, 351K or 351R, and wherein the second CH3 domain comprises one or more of the amino acid variants 351E, 351D, 368E or 368D, said heterodimeric antibody further comprising two light chains that have non-identical sequences

In certain aspects, the second CH3 domain comprises 351D and 368E and the first CH3 domain comprises 366K and 351K.

In a further aspect, provided herein is a pharmaceutical composition comprising an isolated heterodimeric protein of the disclosure and a pharmaceutically acceptable carrier. In certain aspects, the heterodimeric protein is obtained by the method of the disclosure.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect or example of the disclosure are to be understood to be applicable to any other aspect, or example described herein unless incompatible therewith.

Various aspects of the disclosure are described in further detail below.

### BRIEF DESCRIPTION OF FIGURES

Aspects of the disclosure are further described hereinafter with reference to the accompanying drawings, in which:

FIG. 1. Top part: SDS/PAGE labChip results of reactions #1-#12 of Table 2 without FAE. Bottom part: reactions #1-#12 of Table 2 after FAE.

FIG. 2. HP-CIEX results of reactions #1-#12 of Table 2 without FAE and reactions #1-#12 of Table 2 after FAE. FIGS. 2a-2f: upper two graphs show results from reactions without FAE, lower two graphs show results from reactions with FAE. FIG. 2a: Left two graphs show results from #1, right two graphs shows results from #2. FIG. 2b: Left and right two graphs show results from #3, and #4, respectively. FIG. 2c: Left and right two graphs show results from #5, and #6, respectively. FIG. 2d: Left and right two graphs show results from #7, and #8, respectively. FIG. 2e: Left and right two graphs show results from #9, and #10, respectively. FIG. 2f: Left and right two graphs show results from #11, and #12, respectively.

FIG. 3. HP-SEC results of reactions #1-#12 of Table 2 without FAE and reactions #1-#12 of Table 2 after FAE. FIGS. 3a-3d: upper three graphs show results from reactions without FAE, lower three graphs show results from reactions with FAE. FIG. 3a: From left to right: results from #1, #5, #6. FIG. 3b: From left to right: results from #3, #9, #10. FIG. 3c: From left to right: results from #2, #7, #8. FIG. 3d: From left to right: results from #4, #11, #12.

FIG. 4. Gel filtration purification and LabChip analysis results of reactions #1-8 from Table 3 following FAE.

FIG. 5. LabChip analyses of gel filtration-purified FAE products of reactions #1-8 from Table 3 of in non-reducing conditions (upper panel) and reducing conditions (lower panel).

FIG. 6. CIEX results of samples obtained prior to FAE and of samples that were obtained after FAE and after gel filtration. FIG. 6a: Results for reactions #1 and #2 from Table 3 showing formation of IgG heterodimer before FAE (upper graphs) and after FAE (lower graphs). FIG. 6b: Results for reactions #3 and #4 Table 3 showing formation of IgG heterodimer before FAE (upper graphs) and after FAE (lower graphs). FIG. 6c: Results for reactions #5 and #6 Table 3 showing formation of IgG heterodimer before FAE (upper graphs) and after FAE (lower graphs). FIG. 6d: Results for reactions #7 and #8 Table 3 showing formation of IgG heterodimer before FAE (upper graphs) and after FAE (lower graphs).

### DETAILED DESCRIPTION

Human immunoglobulin G (IgG) antibodies exist in four subclasses with distinct structural and functional properties. IgGs are composed of two heavy chain-light chains pairs (half-molecules), which are connected via inter-heavy chain disulphide bonds situated in the hinge region.

Provided herein is a method for producing a heterodimeric protein that comprises two distinct IgG CH3 domains that are capable of forming a CH3-CH3 interface. In certain aspects, the method is an in vitro method.

As used herein, the term “heterodimeric protein” refers to a protein comprising two monomers having non-identical polypeptides that are linked, either covalently or non-covalently. One or each of the monomers may be paired with a light chain. Said heterodimeric protein when used as first and second proteins provided as (a) and (b) of the method of the disclosure, comprises a CH3 domain which either comprises a positively charged amino acid residues at positions 351 and 366 or negatively charged amino acid residues at positions 351 and 368. Said heterodimeric protein as obtained by the method of the disclosure comprises a positively charged amino acid residues at positions 351 and 366 and negatively charged amino acid residues at positions 351 and 368.

### Heterodimeric Protein Products

As would be clear to a person skilled in the art, the properties of the heterodimeric protein produced by the method of the disclosure will be dictated by the starting material, that is the first and second proteins provided as (a) and (b) of the method of the disclosure. Merely by way of example, if the first and second proteins are fragments of IgG antibodies (e.g. if they comprise or consist of IgG CH3 domains), the heterodimeric protein obtained by the method will be a heterodimeric fragment of an IgG antibody (e.g. wherein the heterodimeric fragment will comprise or consist of two IgG CH3 domains). By the same token, if the first and second proteins are IgG antibodies or IgG half-bodies, the produced heterodimeric protein will be an IgG antibody. In certain aspects, the first and/or second protein comprise a CH3 domain, a hinge region and a Fab arm. In principle, the present technology allows to produce heterodimers wherein the format of said first and/or second protein can comprise any multimerizing domain, including but not limited to a variable heavy domain, a CH1, CH2 domain, a variable light chain or the like. In certain aspects, a first protein may comprise an antibody binding domain, and the second protein may comprise a cytokine, ligand, scFv or other domain providing therapeutic potential (e.g., a bi- or multi-functional fusion protein).

### Examples of First and Second Proteins are Described Below.

In the context of a heterodimeric protein produced by the method of the disclosure, the term “heterodimeric protein” refers to a protein comprising two monomers having non-identical polypeptides that are linked, either covalently or non-covalently, wherein the two monomers comprise or consist of two distinct IgG CH3 domains. The IgG CH3 domains are distinct due to their polypeptide sequences being different. Specifically, the two distinct IgG CH3 domains differ in amino acids at least at positions 351, 366 and/or 368. In certain aspects, one of the two CH3 domains comprises a positively charged amino acid residue at position 351 and 366, and the second of the two CH3 domains comprises a negatively charged amino acid residue at position 351 and 368. The amino acid residue numbering is according to EU numbering (available from https://www.imgt.org/IMGTScientificChart/Numbering/Hu_IGHGnber.html, last updated on 20 Jan. 2020 at 21:00:03 CET). A CH3 domain that comprises a positively charged amino acid residue at position 351 and 366 is referred to herein as a “351/366 positive CH3 domain” or as a “first CH3 domain”. By the same token, a CH3 domain that comprises a negatively charged amino acid residue at position 351 and 368 is referred to herein as a “351/368 negative CH3 domain”, or as a “second CH3 domain”.

In the present disclosure, when reference is made to a 351/366 positive CH3 domain or a 351/368 negative CH3 domain, this refers to the charge of the side chains of residues 351, 366 and 368 specifically, and not necessarily the overall charge of the whole CH3 domain. It will be appreciated that the heterodimeric proteins produced by the method of the disclosure are heterodimeric at least because they have two distinct CH3 domains.

The term “CH3 domain” as used herein refers to the CH3 domain of an immunoglobulin, specifically an IgG immunoglobulin. The CH3 domain and its sequence are well known in the art. An “IgG immunoglobulin” (also referred to herein as “IgG”, “IgG molecule”, or “IgG antibody”) means a polypeptide belonging to the class of antibodies substantially encoded by recognised in the art immunoglobulin gamma genes. In humans, the IgG immunoglobulin class includes the subclasses IgG1, IgG2, IgG3, and IgG4. Typically, conventional IgG immunoglobulins are heterotetramers having two heavy chains that are held together by disulphide bonds (—S-S—) at the hinge region and two light chains. However, in the art IgG immunoglobulins are often referred to as dimers (for example homodimers or heterodimers). The dimers are formed by two monomers, wherein each monomer comprises a heavy chain and a light chain. The heavy chain and light chain are held together by disulphide bonds (—S-S—). Such a monomer is referred to as a “half-antibody” or “half-body”.

The method of the disclosure produces heterodimeric proteins as a result of the distinct (i.e. 351/366 positive and 351/368 negative) IgG CH3 domains being capable of forming a CH3-CH3 interface. In certain aspects, the two distinct IgG CH3 domains preferentially bind each other, i.e. have a higher propensity to bind each other than to bind another identically charged IgG CH3 domain. In other words, the 351/368 negative IgG CH3 domain described herein may have a higher propensity to bind a 351/366 positive IgG domain described herein than to another 351/368 negative IgG CH3 domain. Equally, the 351/366 positive IgG CH3 domain described herein may have a higher propensity to bind a 351/368 negative IgG CH3 domain described herein than to another 351/366 positive IgG CH3 domain.

The term “CH3-CH3 interface” as used herein, refers to the association between the two distinct CH3 domains that is as a result of interacting amino acid residues, i.e. at least one interaction between an amino acid of a first CH3 domain and an amino acid of a second CH3 domain. Such interaction is for instance via Van der Waals forces, hydrogen bonds, water-mediated hydrogen bonds, salt bridges or other electrostatic forces, attractive interactions between aromatic side chains, the formation of disulphide bonds, or other forces known to one skilled in the art. It will be appreciated that when two distinct CH3 domains form a CH3-CH3 interface, they form a heterodimeric protein (heterodimeric by virtue of at least the distinct sequences of the CH3 domains).

Interactions between two CH3 domains (such as two CH3 domains of two individual heavy chains) are known to play an important role in driving heavy chain dimerization. Thus, CH3 domains direct the association of antibody heavy chains, and it is known that the interface between CH3 domains contains more than 20 contact residues from each chain that play a role in the CH3-CH3 interaction (Deisenhofer J., Biochemistry 1981(20)2361-2370; Miller S., J. Mol. Biol. 1990(216)965-973; Padlan, Advances in Protein Chemistry 1996 (49) 57-133). The CH3 variants of the present disclosure (that is positively charged amino acid residues at position 351 and 366, and negatively charged amino acid residues at position 351 and 368, specific examples of which are discussed in more detail elsewhere in the present disclosure) can thus be used in association with other antibody domains to generate full length antibodies that are either bispecific or monospecific. The specificity of the antibody as defined by the VH/VL combinations typically does not affect the heavy chain dimerization behaviour that is driven by the CH3 domains.

In certain aspects, the IgG CH3 domains of the heterodimeric protein produced by the method of the disclosure do not comprise an arginine at position 409 and/or do not comprise a leucine at position 405. In some examples, the IgG CH3 domains of the heterodimeric protein produced by the method of the disclosure comprise a lysine at position 409 and/or comprise a phenylalanine at position 405 (EU numbering).

The heterodimeric protein produced by the method described herein is a heterodimeric IgG antibody or a heterodimeric fragment of an IgG antibody. In this context, a heterodimeric fragment of an IgG antibody refers to a molecule that comprises at least two distinct IgG CH3 domains. In certain aspects, the fragment may further comprise one or more domain typically present with an IgG antibody (such as CH2, CH1, VH, CL and/or VL) and/or a specific binding moiety.

In certain aspects, the heterodimeric IgG antibody or a heterodimeric fragment of an IgG antibody may comprise two IgG CH3 domains that comprise or is selected from the group consisting of IgG1, IgG2, IgG3, and IgG4 CH3 domains. In certain aspects, each IgG CH3 domain within the heterodimeric IgG antibody or the heterodimeric fragment thereof is a IgG1 CH3 domain. It will be appreciated that each of the monomers forming the heterodimeric IgG antibody or a heterodimeric fragment of an IgG antibody need not comprise an IgG CH3 domain that is of the same subclass. In certain aspects, each of the CH3 domains of the heterodimeric IgG antibody or a heterodimeric fragment of an IgG antibody is of a different subclass. Merely by way of example, one of the monomers may comprise an IgG1 CH3 domain and the other may comprise an IgG2, IgG3 or IgG4 CH3 domain.

In certain aspects, the IgG CH3 domains in the heterodimeric protein produced by the method described herein is human IgG CH3 domains (e.g. human IgG1, IgG2, IgG3, or IgG4 CH3 domains). In certain aspects, the IgG CH3 domains in the heterodimeric IgG antibody or a heterodimeric fragment of an IgG antibody produced by the method described herein is human IgG CH3 domains (e.g. human IgG1, IgG2, IgG3, or IgG4 CH3 domains).

In a specific example, the heterodimeric protein produced by the method described herein is a heterodimeric IgG antibody. In this specific example, the antibody is a IgG1, IgG2, IgG3, or a IgG4 antibody. In certain aspects, it is a human IgG1. In certain aspects, it is a human IgG2. In certain aspects, it is a human IgG3. ln certain aspects, it is a human IgG4 antibody.

In another specific example, the heterodimeric protein produced by the method described herein is a heterodimeric fragment of an IgG antibody. In this specific example, the heterodimeric fragment is a heterodimeric fragment of a IgG1, IgG2, IgG3, or IgG4 antibody. In certain aspects, it is a heterodimeric fragment of a human IgG1, IgG2, IgG3, and IgG4 antibody.

In certain aspects, when the heterodimeric protein produced by the method described herein is a heterodimeric fragment of an IgG antibody, the fragment may comprise two IgG CH3 domains and two IgG CH2 domains (e.g. each monomer within the heterodimeric protein may comprise an IgG CH3 and an IgG CH2 domain).

In a specific example, the heterodimeric protein produced by the method described herein may comprise or consist of an IgG Fc region. In this specific example, the IgG Fc region is an IgG Fc region of an IgG1, IgG2, IgG3, or IgG4 antibody. In certain aspects, it is a IgG Fc region of a human IgG1, IgG2, IgG3, and IgG4 antibody.

The term “IgG Fc region” as used herein refers to the fragment crystallizable C-terminal region of an immunoglobulin heavy chain. The human IgG heavy chain Fc region is generally defined as comprising the amino acid residue from P230 to the carboxyl-terminus of the IgG antibody. The numbering of residues in the Fc region is that of the EU index. In certain aspects, the Fc region may include the hinge region. The hinge region (e.g., for IgG 1 is residues 216-230 according to the EU numbering) may extend from the N-terminus of the Fc region. Two monomeric IgG Fc domains are held together by disulphide bonds (—S-S—) at the hinge region, aiding the formation and/or maintenance of the heterodimeric protein. The number of hinge disulphide bonds varies among the immunoglobulin subclasses (Papadea and Check 1989). In vivo, an Fc region dimer or heterodimer interacts with the complement system and with specific receptors on the surface of a variety of cells. In certain aspects, the IgG Fc region may comprise the IgG1 or IgG2 core hinge region CPPC. Alternatively, the IgG Fc region may comprise the IgG3 core hinge region CPRC. Alternatively, the IgG Fc region may comprise the IgG4 core hinge region CPSC. The term “core hinge region” as used herein refers to the four amino acids corresponding to positions 226-229 (EU numbering) of a human IgG1 antibody.

In certain aspects, the first protein and the second protein comprise an identical hinge region. In certain aspects, the first protein and the second protein comprise the IgG1 hinge region. In certain aspects, the first protein and the second protein comprise the IgG2 hinge region. In certain aspects, the first protein and the second protein comprise the IgG3 hinge region. In certain aspects, the first protein and the second protein comprise the IgG4 hinge region.

In certain aspects, the heterodimeric protein produced by the method described herein is a heterodimeric antibody. Antibodies produced by the method described herein can have sequences of any origin, including murine and human sequences. Antibodies can consist of sequences from one origin only, such as fully human antibodies, or they can have sequences of more than one origin, resulting for instance in chimeric or humanized antibodies. It is desirable for antibodies for therapeutic uses to be as close to natural antibodies of the subject to be treated as possible (for instance human antibodies for human subjects).

Antibody binding can be expressed in terms of specificity and affinity. The specificity determines which antigen or epitope thereof is bound by the binding domain. The affinity is a measure for the strength of binding to a particular antigen or epitope.

The term “antigen” as used herein means a substance or molecule that, when introduced into the body, triggers the production of an antibody by the immune system. An antigen, among others, is derived from pathogenic organisms, tumour cells or other aberrant cells, from haptens, or even from self-structures. At the molecular level, an antigen is characterized by its ability to be bound by the antigen-binding site of an antibody. Also mixtures of antigens can be regarded as “antigen”, i.e. the skilled person would appreciate that sometimes a lysate of tumour cells, or viral particles is indicated as “antigen” whereas such tumour cell lysate or viral particle preparation exists of many antigenic determinants. An antigen comprises at least one, but often more, epitopes. The term “epitope” as used herein means a part of an antigen that is recognized by the immune system, specifically by antibodies, B cells, or T cells. Although epitopes are usually thought to be derived from non-self-proteins, sequences derived from the host that can be recognized are also classified as epitopes.

It will be appreciated that the heterodimeric protein produced by the method described herein (for example a heterodimeric IgG antibody or a heterodimeric fragment thereof) will be made up of two monomers, wherein each monomer comprises or consists of a distinct IgG CH3 domain (i.e. 351/366 positive IgG CH3 domain and a 351/368 negative IgG CH3 domain). In addition, further distinctions (i.e. differences in sequence) in one or both of the monomers (for example half-bodies or fragments thereof) may also be present.

In certain aspects, these distinctions may give rise to a heterodimeric protein (such as an IgG antibody or heterodimeric fragment thereof) that is multivalent and/or multispecific. The term “multivalent” (for example multivalent antibody or a heterodimeric fragment thereof) refers to a single molecule with more than one valency, where “valency” is described as the number of antigen-binding moieties present per molecule (for example antibody or heterodimeric fragment thereof). As such, the single binding molecule can bind to more than one binding site on a target antigen. Examples of multivalent antibodies include, but are not limited to bivalent antibodies, trivalent antibodies, tetravalent antibodies, pentavalent antibodies, and the like, but including at least antibodies having six valences.

The term “multispecific” (for example multispecific antibody or a heterodimeric fragment thereof) as used herein, refers to a single molecule that binds to two or more different epitopes on at least two or more different antigens. The term “multispecific antibody” includes, but is not limited to bispecific antibodies, trispecific antibodies, tetraspecific antibodies, and the like. In certain aspects, the term “multispecific antibody” refers to a bispecific antibody. In certain aspects, the term “multispecific antibody” refers to a trispecific antibody. In certain aspects, the term “multispecific antibody” refers to an antibody having valences of four, five or six. In certain aspects, the term “multispecific antibody” refers to an antibody having more than six valences.

In certain aspects, the heterodimeric protein produced by the method described herein is a heterodimeric IgG antibody, wherein the antibody is multivalent and/or multispecific.

In certain aspects, the heterodimeric protein (such as an IgG antibody or heterodimeric fragment thereof) may comprise two or more variable regions. In certain aspects, each of those variable regions may specifically bind a different epitope. In certain aspects, the different epitopes are located on different antigens. In certain aspects, the different antigens is expressed on the same or on different cells.

In certain aspects, the heterodimeric antibody may comprise two, non-identical light chains. Alternatively, the heterodimeric antibody may comprise two identical light chains.

In an aspect where the heterodimeric protein produced by the method of the disclosure is an IgG antibody, the antibody may comprise two monomers of the same IgG subclass, for example it may comprise two IgG1 monomers, two IgG2 monomers, two IgG3 monomers or two IgG4 monomers.

In certain aspects, the heterodimeric protein (such as an IgG antibody or heterodimeric IgG fragment thereof) produced by the present disclosure may comprise two identical light chains. Alternatively, the heterodimeric protein (such as an IgG antibody or heterodimeric IgG fragment thereof) produced by the present disclosure may comprise two non-identical light chains.

In certain aspects, provided herein is a method for producing a DEKK heterodimeric protein that comprises two distinct IgG CH3 domains that are capable of forming a CH3-CH3 interface, said method comprising the steps of:
providing:
   (a) a first protein comprising a first CH3 domain which comprises a positively charged amino acid residue at position 351 and 366, wherein the positively charged amino acid residues at position 351 and 366 are K and K, respectively, and
   (b) a second protein comprising a second CH3 domain which comprises a negatively charged amino acid residue at position 351 and 368, wherein the negatively charged amino acid residues at position 351 and 368 are D and E, respectively
   wherein the numbering is according to EU numbering,
incubating the proteins of (a) and (b) together under reducing conditions to provide a reduced first protein and a reduced second protein; and
reoxidising the reduced first and second proteins to obtain the DEKK heterodimeric protein.

In certain aspects, provided herein is a method for producing a heterodimeric antibody that comprises two distinct IgG CH3 domains that are capable of forming a CH3-CH3 interface, said method comprising the steps of:
providing:
   (a) a first antibody or half-body, wherein each CH3 domain comprises a positively charged amino acid residue at position 351 and 366, and
   (b) a second antibody or half-body, wherein each CH3 domain comprises a negatively charged amino acid residue at position 351 and 368,
   wherein the numbering is according to EU numbering,
incubating the antibodies and/or half-bodies of (a) and (b) together under reducing conditions to provide a reduced first antibody or half-body and a reduced second antibody or half-body; and
reoxidising the reduced first antibodies and/or second half-bodies to obtain the heterodimeric antibody.

In certain aspects, provided herein is a method for producing an IgG heterodimeric antibody that comprises two distinct IgG CH3 domains that are capable of forming a CH3-CH3 interface, said method comprising the steps of:
providing:
   (a) a first IgG antibody or IgG half-body, wherein each CH3 domain comprises a positively charged amino acid residue at position 351 and 366, and
   (b) a second IgG antibody or IgG half-body, wherein each CH3 domain comprises a negatively charged amino acid residue at position 351 and 368,
   wherein the numbering is according to EU numbering,
incubating the IgG antibodies and/or IgG half-bodies of (a) and (b) together under reducing conditions to provide a reduced first IgG antibody or IgG half-body and a reduced second IgG antibody or IgG half-body; and
reoxidising the reduced IgG antibodies and/or IgG half-bodies to obtain the heterodimeric IgG antibody.

In certain aspects, provided herein is a method for producing a DEKK heterodimeric antibody that comprises two distinct IgG CH3 domains that are capable of forming a CH3-CH3 interface, said method comprising the steps of:
providing:
   (a) a first antibody or half-body, wherein each CH3 domain comprises a positively charged amino acid residue at position 351 and 366, wherein the positively charged amino acid residues at position 351 and 366 are K and K, respectively, and
   (b) a second antibody or half-body, wherein each CH3 domain comprises a negatively charged amino acid residue at position 351 and 368, wherein the negatively charged amino acid residues at position 351 and 368 are D and E, respectively
   wherein the numbering is according to EU numbering,
incubating the antibodies and/or half-bodies of (a) and (b) together under reducing conditions to provide a reduced first antibody or half-body and a reduced second antibody or half-body; and
reoxidising the reduced antibodies and/or half-bodies to obtain the DEKK heterodimeric antibody.

In certain aspects, provided herein is a method for producing a DEKK heterodimeric IgG antibody that comprises two distinct IgG CH3 domains that are capable of forming a CH3-CH3 interface, said method comprising the steps of:
providing:
   (a) a first IgG antibody or IgG half-body, wherein each CH3 domain comprises a positively charged amino acid residue at position 351 and 366, wherein the positively charged amino acid residues at position 351 and 366 are K and K, respectively, and
   (b) a second IgG antibody or IgG half-body, wherein each CH3 domain comprises a negatively charged amino acid residue at position 351 and 368, wherein the negatively charged amino acid residues at position 351 and 368 are D and E, respectively
   wherein the numbering is according to EU numbering,
incubating the IgG antibodies and/or IgG half-bodies of (a) and (b) together under reducing conditions to provide a reduced first IgG antibody or IgG half-body and a reduced second IgG antibody or IgG half-body; and
reoxidising the reduced IgG antibodies and/or IgG half-bodies to obtain the DEKK heterodimeric IgG antibody.

### Methods for Making Heterodimeric Proteins

The method of the present disclosure comprises the step of providing: (a) a first protein comprising a CH3 domain which comprises a positively charged amino acid residue at position 351 and 366 (also referred to herein as a 351/366 positive CH3 domain), and (b) a second protein comprising a CH3 domain which comprises a negatively charged amino acid residue at position 351 and 368 (also referred to herein as a 351/368 negative CH3 domain), wherein the numbering is according to EU numbering.

In certain aspects, the first and/or second proteins may comprise or is selected from the group consisting of: a monomeric protein, a homodimeric protein and a heterodimeric protein.

It will be understood by a person skilled in the art that the terms “monomeric protein” and “monomer”, which are used interchangeably herein, generally refer to a single, non-aggregated protein or polypeptide molecule. However, as mentioned, in the field of antibodies, the term monomer may also refer to a half-body. For example an IgG monomer is an IgG half-body, i.e. a molecule which comprises or consists of one IgG heavy chain linked to one IgG light chain. The one light chain and the one heavy chain is linked by a disulphide bond in the IgG half-body. Accordingly in the context of the present disclosure, the term monomer, depending on the context, means a single, non-aggregated protein or polypeptide molecule (such as a single IgG CH3 domain or a monomeric IgG Fc region, for example), or a half-body (e.g. IgG half-body).

Similarly, in the art the terms “homodimeric protein” or “homodimer” generally refer to a dimer formed from two identical polypeptides (for example two 351/368 negative CH3 domains or two 351/366 positive CH3 domains) that are linked, either covalently or non-covalently. However, in the field of antibodies, the term homodimer may also refer to an antibody (such as an IgG antibody) with two identical half-bodies. Therefore, in the context of the present disclosure, the term homodimer, depending on the context, means a dimer formed from two identical polypeptides (for example two identical IgG CH3 domains), linked either covalently or non-covalently, or a homodimeric antibody (e.g. homodimeric IgG antibody).

Accordingly, when the first protein is a homodimer, the method of the present disclosure comprises the step of providing: (a) a first protein (e.g. a homodimeric IgG antibody or a fragment thereof) comprising two (identical) CH3 domains which each comprise a positively charged amino acid residue at position 351 and 366 (also referred to herein as a 351/366 positive CH3 domain). Similarly, when the second protein is a homodimer, the method of the present disclosure comprises the step of providing (b) a second protein (e.g. a homodimeric IgG antibody or a fragment thereof) comprising two (identical) CH3 domains which each comprise a negatively charged amino acid residue at position 351 and 368 (also referred to herein as a 351/368 negative CH3 domain).

As stated elsewhere herein, the term “heterodimeric protein” or “heterodimer” refers to a protein comprising two monomers having non-identical polypeptides that are linked, either covalently or non-covalently. In the context of the first and second proteins described herein, it refers to a protein comprising two monomers that are distinct (i.e. have non-identical polypeptide sequences) but comprise IgG CH3 domains that have the same charge (i.e. the CH3 domains in the first protein heterodimer are both 351/366 positive IgG CH3 domains and CH3 domains in the second protein heterodimer are both 351/368 negative IgG CH3 domains). Accordingly, when the first protein is a heterodimer, the method of the present disclosure comprises the step of providing: (a) a first protein (e.g. a heterodimeric IgG antibody or a fragment thereof) comprising two CH3 domains which each comprise a positively charged amino acid residue at position 351 and 366 (also referred to herein as a 351/366 positive CH3 domain). Similarly, when the second protein is a heterodimer, the method of the present disclosure comprises the step of providing (b) a second protein (e.g. a heterodimeric IgG antibody or a fragment thereof) comprising two CH3 domains which each comprise a negatively charged amino acid residue at position 351 and 368 (also referred to herein as a 351/368 negative CH3 domain).

In certain aspects, the two IgG CH3 domains of the two monomers forming the heterodimer of the first protein have the same amino acids at positions 351 and 366, and similarly, the two IgG CH3 domains of the two monomers forming the heterodimer of the second protein have the same amino acids at positions 351 and 368. In certain aspects, the IgG CH3 domains of the two monomers forming the heterodimer is identical (i.e. the polypeptide sequences of the IgG CH3 domains is the same along their entire lengths).

In the field of antibodies, the term heterodimer may also refer to an antibody (such as an IgG antibody) comprising or consisting of non-identical half-bodies. Therefore, in the context of the present disclosure, the term heterodimeric protein, may also mean a heterodimeric antibody. Such a heterodimeric antibody is made from two non-identical half-bodies, wherein the two half-bodies comprise IgG CH3 domains that have the same charge (i.e. 351/366 positive or 351/368 negative IgG CH3 domains). In certain aspects, the IgG CH3 domains of the two half-bodies forming the heterodimeric antibody have the same amino acids at positions 351 and 366, or 351 and 368 (depending on whether the IgG CH3 domains are 351/366 positive or 351/368 negative). In certain aspects, the IgG CH3 domains of the two half-bodies forming the heterodimeric antibody is identical (i.e. the polypeptide sequences of the IgG CH3 domains is same along their entire lengths).

In certain aspects, the first and second proteins provided in (a) and (b) of the method of the disclosure is monomers (for example, the first and second proteins is first and second half-bodies, respectively). In certain aspects, the first protein (for example the first half-body) may comprise a 351/366 positive IgG CH3 domain, and the second protein (for example the second half-body) may comprise a 351/368 negative IgG CH3 domain. Upon completion of reduction and reoxidising steps of the method of the disclosure, the heterodimeric protein (for example a heterodimeric antibody) produced will comprise the first and second proteins (for example first and second half-bodies).

In another example, the first protein provided in (a) of the method of the disclosure is a monomer (for example, the first protein is a half-body) and the second protein provided as (b) is a homodimeric protein (for example, the second protein is a homodimeric antibody). The monomeric protein (for example the half-body), may comprise a 351/366 positive IgG CH3 domain and the homodimeric protein (for example a homodimeric antibody) may comprise two 351/368 negative IgG CH3 domains. Upon completion of steps ii) and iii) of the method of the disclosure, the heterodimeric protein (for example a heterodimeric antibody) produced will comprise the monomeric protein (which is a half-body) and one of the monomers of the homodimeric protein (for example one of the half-bodies of the homodimeric antibody). Therefore, the produced heterodimeric protein (for example heterodimeric antibody) will comprise a 351/366 positive IgG CH3 domain and a 351/368 negative IgG CH3 domain.

In another example, the first protein provided in (a) of the method of the disclosure is a homodimeric protein (for example, the first protein is a homodimeric antibody) and the second protein provided in (b) is a monomer (for example, the second protein is a half-body). The homodimeric protein (for example a homodimeric antibody) may comprise two 351/366 positive IgG CH3 domains, and the monomeric protein (for example the half-body) may comprise a 351/368 negative IgG CH3 domain. Upon completion of steps ii) and iii) of the method of the disclosure, the heterodimeric protein (for example a heterodimeric antibody) produced will comprise one of the monomers of the homodimeric protein (for example one of the half-bodies of the homodimeric antibody) and the monomeric protein (which is a half-body). Therefore, the produced heterodimeric protein (for example heterodimeric antibody) will comprise a 351/366 positive IgG CH3 domain and a 351/368 negative IgG CH3 domain.

In another example, the first protein provided in (a) of the method of the disclosure is a homodimeric protein (for example, a first homodimeric antibody) and the second protein provided in (b) may also be a homodimeric protein (for example, a second homodimeric antibody). The first homodimeric protein (for example the first homodimeric antibody) may comprise two 351/366 positive IgG CH3 domains, and the second protein (for example the second homodimeric antibody) may comprise two 351/368 negative IgG CH3 domains. Upon completion of reducing and reoxidising steps of the method of the disclosure, the heterodimeric protein (for example a heterodimeric antibody) produced will comprise one of the monomers of the first homodimeric protein (for example one of the half-bodies of the first homodimeric antibody) and one of the monomers of the second homodimeric protein (for example one of the half-bodies of the second homodimeric antibody). Therefore, the produced heterodimeric protein (for example heterodimeric antibody) will comprise a 351/366 positive IgG CH3 domain and a 351/368 negative IgG CH3 domain.

In another example, the first protein provided in (a) of the method of the disclosure is a monomer (for example, the first protein is a half-body) and the second protein provided in (b) is a heterodimeric protein (for example, the second protein is a heterodimeric antibody). The monomeric protein (for example the half-body), may comprise a 351/366 positive IgG CH3 domain and the heterodimeric protein (for example a heterodimeric antibody) may comprise a 351/368 negative IgG CH3 domain. Upon completion of reducing and reoxidising steps of the method of the disclosure, the heterodimeric protein (for example a heterodimeric antibody) produced will comprise the monomeric protein (which is a half-body) and one of the monomers of the heterodimeric protein (for example one of the half-bodies of the homodimeric antibody). Therefore, the produced heterodimeric protein (for example heterodimeric antibody) will comprise a 351/366 positive IgG CH3 domain and a 351/368 negative IgG CH3 domain.

In another example, the first protein provided in (a) of the method of the disclosure is a heterodimeric protein (for example, the first protein is a heterodimeric antibody) and the second protein provided in (b) is a monomer (for example, the second protein is a half-body). The heterodimeric protein (for example a heterodimeric antibody) may comprise two 351/366 positive IgG CH3 domains, and the monomeric protein (for example the half-body) may comprise a 351/368 negative IgG CH3 domain. Upon completion of reducing and reoxidising steps of the method of the disclosure, the heterodimeric protein (for example a heterodimeric antibody) produced will comprise one of the monomers of the heterodimeric protein (for example one of the half-bodies of the heterodimeric antibody) and the monomeric protein (which is a half-body). Therefore, the produced heterodimeric protein (for example heterodimeric antibody) will comprise a 351/366 positive IgG CH3 domain and a 351/368 negative IgG CH3 domain.

In another example, the first protein provided in (a) of the method of the disclosure is a heterodimeric protein (for example, a first heterodimeric antibody) and the second protein provided in (b) may also be a homodimeric protein (for example, a second homodimeric antibody). The first heterodimeric protein (for example the first heterodimeric antibody) may comprise two 351/366 positive IgG CH3 domains, and the second protein (for example the second homodimeric antibody) may comprise two 351/368 negative IgG CH3 domains. Upon completion of reducing and reoxidising steps of the method of the disclosure, the heterodimeric protein (for example a heterodimeric antibody) produced will comprise one of the monomers of the first heterodimeric protein (for example one of the half-bodies of the first heterodimeric antibody) and one of the monomers of the second homodimeric protein (for example one of the half-bodies of the second homodimeric antibody). Therefore, the produced heterodimeric protein (for example heterodimeric antibody) will comprise a 351/366 positive IgG CH3 domain and a 351/368 negative CH3 domain.

In another example, the first protein provided in (a) of the method of the disclosure is a homodimeric protein (for example, a first homodimeric antibody) and the second protein provided in (b) may also be a heterodimeric protein (for example, a second heterodimeric antibody). The first homodimeric protein (for example the first homodimeric antibody) may comprise two 351/366 positive IgG CH3 domains, and the second protein (for example the second heterodimeric antibody) may comprise two 351/368 negative IgG CH3 domains. Upon completion of reducing and reoxidising steps of the method of the disclosure, the heterodimeric protein (for example a heterodimeric antibody) produced will comprise one of the monomers of the first homodimeric protein (for example one of the half-bodies of the first homodimeric antibody) and one of the monomers of the second heterodimeric protein (for example one of the half-bodies of the second heterodimeric antibody). Therefore, the produced heterodimeric protein (for example heterodimeric antibody) will comprise a 351/366 positive IgG CH3 domain and a 351/368 negative CH3 domain.

In another example, the first protein provided in (a) of the method of the disclosure is a heterodimeric protein (for example, a first heterodimeric antibody) and the second protein provided in (b) may also be a heterodimeric protein (for example, a second heterodimeric antibody). The first heterodimeric protein (for example the first heterodimeric antibody) may comprise two 351/366 positive IgG CH3 domains, and the second protein (for example the second heterodimeric antibody) may comprise two 351/368 negative IgG CH3 domains. Upon completion of reducing and reoxidising steps of the method of the disclosure, the heterodimeric protein (for example a heterodimeric antibody) produced will comprise one of the monomers of the first heterodimeric protein (for example one of the half-bodies of the first heterodimeric antibody) and one of the monomers of the second heterodimeric protein (for example one of the half-bodies of the second heterodimeric antibody). Therefore, the produced heterodimeric protein (for example heterodimeric antibody) will comprise a 351/366 positive IgG CH3 domain and a 351/368 negative CH3 domain.

In certain aspects, in an aspect where the first and/or second protein provided in (a) of the method is a monomeric protein, it is a monomeric Fc region.

In certain aspects, in an aspect where the first and/or second protein provided in (a) of the method is a dimer (a heterodimer or a homodimer), it is a dimeric Fc region.

In certain aspects, the first protein and/or the second protein may comprise or is selected from the group consisting of: an antibody and a half-antibody, or a fragment thereof. Antibodies, half-bodies and fragments thereof are described elsewhere herein. In certain aspects, the fragment is a monomeric or dimeric Fc region.

In certain aspects, the first and/or second proteins can have sequences of any origin, for example murine and human sequences. The first protein and/or the second protein can consist of sequences from one origin only, such as fully human antibodies, or they can have sequences of more than one origin, resulting for instance in chimeric or humanized antibodies. It may be desirable for the first protein and/or the second protein (such as first and/or second antibody) for therapeutic to be as close to natural antibodies of the subject to be treated as possible (for instance human antibodies for human subjects). Accordingly, in certain aspects, the antibody, half-antibody, or fragment thereof is a human antibody, human half-antibody, or fragment thereof. It will be appreciated that the fragment must at least comprise an IgG CH3 domain, a hinge region and a Fab arm. In certain aspects, said Fab arm comprises a variable heavy domain. In certain aspects, said Fab arm comprises a variable heavy domain and a variable light chain. In certain aspects, said Fab arm comprises a variable heavy domain and does not comprise (i.e. lacks) a variable light chain.

In certain aspects, when the first and/or second protein is an antibody, it is a homodimeric antibody or a heterodimeric antibody.

In certain aspects, the first protein is an antibody having a first binding specificity and the second protein is an antibody have a second distinct binding specificity.

In certain aspects, the first protein may be an antibody comprising a binding specificity and the second protein may be an antibody comprising another distinct binding specificity.

As mentioned above, the production of a heterodimeric protein according to the method described herein is facilitated by the preferential binding of a 351/366 positive CH3 domain (which comprises a positively charged amino acid residue at position 351 and 366) to a 351/368 negative CH3 domain (which comprises a negatively charged amino acid residue at position 351 and 368).

In certain aspects, the 351/366 positive CH3 domain may comprise amino acids K or R at position 351, and amino acids K or R at position 366. For example, the 351/366 positive CH3 domain may comprise amino acid K at position 351 and amino acid R at position 366, or amino acid R at position 351 and amino acid K at position 366, or amino acid K at position 351 and amino acid K at position 366, or amino acid R at position 351 and amino acid R at position 366. In certain aspects, the 351/366 positive CH3 domain may comprise amino acid K at position 351 (i.e. may comprise 351K) and amino acid K at position 366 (i.e. may comprise 366K). A first protein comprising two of the latter 351/366 positive CH3 domains is referred to as a KKKK first protein herein. In certain aspects, the KKKK first protein is a homodimer or a heterodimer. In certain aspects, the KKKK first protein is a homodimeric IgG antibody or fragment thereof or a heterodimeric IgG antibody or fragment thereof. In certain aspects, the KKKK IgG antibody or fragment thereof is an IgG1, IgG2, IgG3 or IgG4 antibody or fragment thereof.

In certain aspects, the 351/368 negative CH3 domain may comprise amino acids D or E at position 351, and amino acids D or E at position 368. For example, the 351/368 negative CH3 domain may comprise amino acid D at position 351 and amino acid E at position 368, or amino acid E at position 351 and amino acid D at position 368, or amino acid E at position 351 and amino acid E at position 368, or amino acid D at position 351 and amino acid D at position 368. In certain aspects, the 351/368 negative CH3 domain may comprise amino acid D at position 351 (i.e. may comprise 351D) and amino acid E at position 368 (i.e. may comprise 368E). A second protein comprising two of the latter 351/368 negative CH3 domains is referred to as a DEDE second protein herein. In certain aspects, the DEDE second protein is a homodimer or a heterodimer. In certain aspects, the DEDE second protein is a homodimeric IgG antibody or fragment thereof or a heterodimeric IgG antibody or fragment thereof. In certain aspects, the DEDE IgG antibody or fragment thereof is an IgG1, IgG2, IgG3 or IgG4 antibody or fragment thereof.

In certain aspects, the 351/366 positive CH3 domain may comprise amino acid K at position 351 (i.e. may comprise 351K) and amino acid K at position 366 (i.e. may comprise 366K) and the 351/368 negative CH3 domain may comprise amino acid D at position 351 (i.e. may comprise 351D) and amino acid E at position 368 (i.e. may comprise 368E). A heterodimer with one 351/366 positive CH3 domain and one 351/368 negative CH3 domain as described above is referred to as a DEKK heterodimer herein. In certain aspects, the DEKK heterodimer is a heterodimeric IgG antibody or fragment thereof. In certain aspects, the DEKK IgG antibody or fragment thereof is an IgG1, IgG2, IgG3 or IgG4 antibody or fragment thereof.

It will be appreciated that the above mentioned amino acid positions are based on a human IgG CH3 domain. However, in an aspect where a non-human IgG CH3 domain is utilised, the same amino acid substitutions are introduced to corresponding amino acid residues.

In certain aspects, the first and second proteins is provided at a ratio that is favourable for the production of the heterodimeric protein by the method described herein. By “favourable” it means it increases the proportion of heterodimeric proteins produced (where the heterodimeric proteins have a 351/366 positive CH3 domain and a 351/368 negative CH3 domain), as compared to when an equal amount of first and second proteins is provided. In certain aspects, the ratio of the first protein (comprising a 351/366 positive IgG CH3 domain) to the second protein (comprising a 351/368 negative IgG CH3 domain) is between 20:1 and 1:20 (w/w). Ratios going beyond these amounts may be used, while practically may lead to less efficient use of protein. In certain aspects, said ratio is between 10:1 and 1:10. In certain aspects, said ratio is between 5:1 and 1:5. In certain aspects, said ratio is at least 1:1 (w/w), for example at least 1.2:1 (w/w), for example, at least 1.5:1 (w/w) or at least 2:1 (w/w). In certain aspects, the ratio of the first protein (comprising a 351/366 positive IgG CH3 domain) to the second protein (comprising a 351/368 negative IgG CH3 domain) is at between 1:1 (w/w) and 2:1 (w/w).

In certain aspects, the ratio of a KKKK first protein (comprising a 351/366 positive IgG CH3 domain) to a DEDE second protein (comprising a 351/368 negative IgG CH3 domain) is at least 1:1 (w/w), for example at least 1.2:1 (w/w), for example, at least 1.5:1 (w/w) or at least 2:1 (w/w).

In certain aspects, the ratio of the KKKK first protein (comprising a 351/366 positive IgG CH3 domain) to the DEDE second protein (comprising a 351/368 negative IgG CH3 domain) is at between 1:1 (w/w) and 2:1 (w/w).

In certain aspects, the ratio of a KK first protein (comprising a 351/366 positive IgG CH3 domain) to a DEDE second protein (comprising a 351/368 negative IgG CH3 domain) is between 20:1 and 1:20 (w/w). In certain aspects, said ratio is between 10:1 and 1:10. In certain aspects, said ratio is between 5:1 and 1:5. In certain aspects, said ratio is at least 1:1 (w/w), for example at least 1.2:1 (w/w), for example, at least 1.5:1 (w/w) or at least 2:1 (w/w). In certain aspects, the ratio of the KK first protein (comprising a 351/366 positive IgG CH3 domain) to the DEDE second protein (comprising a 351/368 negative IgG CH3 domain) is at between 1:1 (w/w) and 2:1 (w/w).

It will be appreciated that any appropriate amount of the first protein and second protein is used when incubating said first and second proteins under reducing conditions, followed by reoxidising them. Appropriate concentrations is determined by a person of skill in the art, e.g. using the methodology described in the examples section below. As a non-limiting example, each of the first and second proteins are used under said reducing conditions at a concentration of at least 50 μg/ml, for example at least 0.1 mg/ml, at least 1.0 mg/ml, at least 10 mg/ml or at least 25 mg/ml but not more than 100 mg/ml. In certain aspects, when each of the first and second proteins is an antibody, the first and second proteins are each used under said reducing conditions at a concentration of at least 50 μg/ml, for example at least 0.1 mg/ml or at least 1.0 mg/ml. Typically, they may each be used at a concentration range of from 50 μg/ml to 2 mg/ml, e.g. 50 μg/ml to mg/ml. Merely by way of example, they each may be used at a concentration of about 1.1 mg/ml. It will also be appreciated that additional reagents may be present under said reducing conditions to facilitate the process of obtaining reduced first proteins and reduced second proteins. For example in a certain aspect, cystamine is added at an appropriate concentration e.g. in the range of 2 to 70 mM under the reducing conditions to further reduce any homodimers present within the reaction. For example, when the first and second proteins are antibodies (such as homodimeric antibodies), cystamine is added at a concentration of between about 2 to about 70 mM under the reducing conditions. In certain aspects, cystamine is added in the range of 2 to 32 mM under the reducing conditions to further reduce any homodimers present within the reaction. For example, when the first and second proteins are antibodies (such as homodimeric antibodies), cystamine is added at a concentration of between about 2 to about 32 mM under the reducing conditions.

In certain aspects, the first and second proteins are obtained independently from each other. Merely by way of example, the first and second proteins are produced by different host cells. Exemplary method of obtaining a first and second protein are provided in the Examples section below. However, other methods will be known to those skilled in the art.

A “host cell” may be any host cell capable of expressing recombinant DNA molecules and expressing binding moieties known in the art.

In certain aspects, the first protein (such as a homodimeric antibody) and/or second protein (such as a homodimeric antibody) are obtained under serum free conditions (for example by culturing host cells in FreeStyle 293 medium or FreeStyle Cho medium, Invitrogen).

In certain aspects, between providing said first and second proteins in (a) and (b), and incubation under reducing conditions of the method, the first and/or second proteins are purified using methods known in the art. Such methods may include precipitation, centrifugation, filtration, size-exclusion chromatography, affinity chromatography, cation- and/or anion-exchange chromatography, hydrophobic interaction chromatography, and the like. For a mixture of antibodies comprising IgG molecules, protein A or protein G affinity chromatography can be used (see e.g. U.S. Pat. Nos. 4,801,687 and 5,151,504).

Said incubation under reducing and reoxidising conditions of the method facilitate the production of the heterodimeric proteins by recombination of the first and second proteins (such as antibodies, or dimeric fragments thereof), or binding of the first and second proteins (such as half-bodies or monomeric fragments thereof). Recombination of the first and second protein occurs when the first and second proteins exchange monomers (for example half-bodies or dimeric fragments thereof) to produce a heterodimeric protein comprising two distinct IgG CH3 domains that are capable of forming a CH3-CH3 interface.

The term “incubate” or “incubating” as used herein refers to holding, keeping or maintaining first and second proteins together under the relevant conditions (i.e. reducing conditions). The first and second proteins are incubated together within a composition or preparation comprising the first and second proteins.

Said incubation under reducing conditions of the method comprises incubating the proteins of (a) and (b) together under reducing conditions to provide a reduced first protein and/or a reduced second protein.

The term “reducing conditions” refers to an environment in which the first and/or second protein is more likely to become reduced than oxidised. In certain aspects, the reducing conditions may result in “disulphide bond reduction” (i.e. the process of cleaving a disulphide bond, thereby resulting in two thiol groups (—SH groups)). It will be appreciated by a person of skill in the art that when an antibody (homodimeric or heterodimeric antibody) is incubated under reducing conditions, the reduction of disulphide bonds (such as those in the core hinge region), may result in the separation of the antibody into two half-bodies.

The step of incubating the first and second proteins under reducing conditions may comprise incubating the first and second proteins in the presence of a reducing agent. The term “reducing agent” refers to a compound which reduces molecules in its environment, i.e., which changes molecules in its environment to become more reduced. A reducing agent may act by donating electrons, thereby becoming itself oxidized after having reduced a substrate (i.e. the first and/or second protein).

Examples of reducing agents include 2-mercaptoethylamine (2-MEA), dithiothreitol (DTT), dithioerythritol (DTE), glutathione, tris(2-carboxyethyl)phosphine (TCEP), L-cysteine, beta-mercapto-ethanol, thioglycolate, cysteamine, homocysteine, penicillamine, and/or sodium borohydride.

In certain aspects, when the reducing agent is 2-MEA, it is at a concentration of from about 25 mM to about 100 mM, for example from about 50 mM to about 75 mM. In certain aspects, the concentration of 2-MEA is about 75 mM.

It will be appreciated that the time of incubation with a reducing agent may depend upon the concentration and/or temperature at which the incubation occurs. For example, a higher concentration of the reducing agent may allow for a shorter incubation time and/or lower incubation temperature.

In certain aspects, when the concentration of 2-MEA is about 75 mM, the incubation is at about 31° C. and last for at least 300 minutes. In certain aspects, when the concentration of 2-MEA is about 75 mM, the incubation is at about 31° C. and last about 300 minutes.

In one aspect, the reducing agent does not comprise an enzyme.

Additionally or alternatively, incubating the first and second proteins under reducing conditions may comprise incubating the first and second proteins at a pH of 6.0 or more, for example at a pH of 7.0 or more, at a pH of 8.0 or more, at a pH of 9.0 or more, at a pH of 10.0 or more, at a pH of 11.0 or more, or at a pH of 12.0. For example the first and second proteins are incubated at a pH of between 6.0 and 11.0, or between a pH of between 6.0 and 10.0, optionally wherein the pH is between 7.0 and 8.0 (for example between pH 7.3 to 7.5). In certain aspects, the pH is 7.4. Additionally or alternatively, incubating the first and second proteins under reducing conditions may comprise incubating the proteins at a redox potential between −150 and −600 mV, optionally wherein the redox potential is between −250 and −400 mV. Examples of suitable reducing conditions are known in the art. Some examples are described in Labrijn A F., Nature Protocols 2014, Vol. 9, No. 10, pp 2450-2463.

The method further comprises reoxidising the reduced proteins to obtain the heterodimeric protein. The term “reoxidising” or “oxidise” as used means to undergo or cause to undergo a reaction in which electrons are lost to another species. In the context of the present disclosure, the step of reoxidising may allow the reduced first and reduced second proteins as obtained to bind together to form heterodimeric proteins comprising two distinct IgG CH3 domains that are capable of forming a CH3-CH3 interface.

Due to the first protein having a 351/366 positive IgG CH3 domain and the second protein having a 351/368 negative IgG CH3 domain, which have preferential binding for each other, a greater proportion of heterodimeric proteins comprising two distinct IgG CH3 domains will be formed than dimeric proteins (homodimeric or heterodimeric) that have identically charged IgG CH3 domains. Therefore, the present disclosure provides methods for the efficient and controlled production of a well-defined mixture of Ig antibodies or heterodimeric fragments thereof, with a high proportion of bispecifics in the mixture. Even a proportion of bispecifics of at least 80%, at least 85%, at least 90%, at least 95%, at least 97% or more may be obtained in a system where bispecifics are desired. This means that only 5% or less, or 3% or less monospecific bivalent by-products are obtained. Of note, the monomeric by-products, i.e. half molecules produced by the method described herein are more stable than monomeric by-products produced by at least some of the others methods known in the art (such as the method described in WO2011131746). This is advantageous as these half-molecules can further subjected to reducing conditions (discussed elsewhere in the present disclosure) in order to produce the desired heterodimeric proteins, without the need of repeating the step of oxidisation.

In certain aspects, reoxidising the reduced proteins is achieved by removing the reducing agent from the mixture of reduced first and reduced second proteins. Merely by way of example, the reducing agent is removed by diafiltration (such as for example described in Labrijn A F., Nature Protocols 2014, Vol. 9, No. 10, pp 2450-2463). It will be appreciated that the reducing agent need not to be completely removed in order for the reduced first and second proteins to be reoxidised. For example in the context of 2-MEA as a reducing agent, reducing the concentration to less 50 μM may be sufficient to reoxidise the reduced first and reduced second protein. Another example of a method for reoxidising the reduced proteins is buffer exchange. In certain aspects, buffer exchange is against PBS. Optionally, the PBS has a pH of 7.4. Methods for performing buffer exchange will be known to a person skilled in the art. In certain aspects, buffer exchange is carried out using Zeba plates, as exemplified in the Examples section of the present disclosure. By way of another example, buffer exchange may be against PBS using ÄKA Pure 25 system with a Spark ALIAS autosampler and a desalting column (such as HiPrep 26/10). In such an example, buffer exchange may occur at 6 mL/min flow rate and 20 degree celsius.

Upon completion of buffer exchange, samples may be kept at a temperature of about 4° C. for about 24, 48, 64, or more hours to allow complete reoxidation.

Alternatively or additionally the step of reoxidising involves incubating the reduced first and reduced second proteins together with an oxidising agent.

In certain aspects, the method further comprises the step of enriching for and/or isolating the heterodimeric protein as obtained. The heterodimeric protein may be enriched and/or isolated from any contaminates that may arise from the method described herein by routine methods, such as routine purification methods. Such contaminants may include the homodimeric proteins (proteins comprising two monomers each having the same IgG CH3 domain), and/or monomeric proteins (for example half-bodies). Methods for purifying the produced heterodimeric protein may include precipitation, centrifugation, filtration, size-exclusion chromatography, affinity chromatography, cation- and/or anion-exchange chromatography, hydrophobic interaction chromatography, and the like. For a mixture of antibodies comprising IgG molecules, protein A or protein G affinity chromatography can be used (see e.g. U.S. Pat. Nos. 4,801,687 and 5,151,504).

In certain aspects, said incubation under reducing conditions and reoxidising the reduced proteins together are also described as incubating the first and second proteins under conditions that are sufficient to allow cysteines in the CH3 regions to undergo disulphide-bond isomerization to obtain the heterodimeric protein. In certain aspects when the first protein and second protein are antibodies or half-antibodies, incubation under reducing conditions and reoxidising the reduced proteins together, is described as incubating the first and second proteins under conditions that are sufficient to allow cysteines in the core hinge region of the first and second proteins to undergo disulphide-bond isomerization to obtain the heterodimeric protein.

In certain aspects, the first protein and/or the second protein comprises a fusion protein. Said fusion protein may comprise an antibody binding domain, a scFv, a ligand, protein receptor or a cytokine. In certain aspects, the first protein and/or the second protein comprises an antibody binding domain, a scFv, a ligand, protein receptor or cytokine. In certain aspects, said heterodimeric protein is a bifunctional or multifunctional fusion protein.

In a further aspect, provided herein is an isolated heterodimeric protein obtainable by the methods of the disclosure.

In certain aspects, the heterodimeric protein obtainable by the methods of the disclosure is an IgG antibody, for example and IgG1, IgG2, IgG3 or IgG4.

In certain aspects, the IgG antibody may comprise two light chains that have non-identical sequences.

In a further aspect, provided herein is an isolated heterodimeric antibody comprising a 351/366 positive IgG CH3 domain and a 351/368 negative IgG CH3 domain, wherein the 351/366 positive CH3 domain and the 351/368 negative CH3 domain are capable of forming a CH3-CH3 interface, and wherein the 351/368 negative CH3 domain comprises one or more of the amino acid variants 351E, 351D, 368E or 368D and said 351/366 positive CH3 domain comprises one or more of the amino acid variants 366K, 366R, 351K or 351R, said heterodimeric antibody further comprising two light chains that have non-identical sequences.

In certain aspects, the 351/368 negative CH3 domain comprises 351D and 368E and the 351/366 positive IgG CH3 domain comprises 366K and 351K.

It will be appreciated that aspects relating to the heterodimeric protein mentioned herein in the context of the method of the disclosure, equally apply to the heterodimeric protein obtainable by the method described herein, and the isolated heterodimeric antibody comprising a 351/366 positive IgG CH3 domain and a 351/368 negative IgG CH3 domain, wherein the 351/366 positive CH3 domain and the 351/368 negative CH3 domain are capable of forming a CH3-CH3 interface, wherein the 351/368 negative CH3 domain comprises one or more of the amino acid variants 351E, 351D, 368E or 368D and said 351/366 positive CH3 domain comprises one or more of the amino acid variants 366K, 366R, 351K or 351R, said heterodimeric antibody further comprising two light chains that have non-identical sequences.

The variations at position 351/366 and/or at 351/368 can be combined with any one of the modifications mentioned in WO2020/226502 A2, which is incorporated herein by reference in its entirety. Equally, variations at position 351/366 and/or at 351/368 can be combined with any one of the modifications mentioned in WO2021/235936 A1, which is incorporated herein by reference in its entirety.

In a further aspect, provided herein is a pharmaceutical composition comprising an isolated heterodimeric protein of the disclosure and a pharmaceutically acceptable carrier. The term “pharmaceutical composition” refers to a preparation that is in such a form that it allows the biological activity of an active ingredient (for example the heterodimeric protein of the disclosure) contained to be effective and that it does not contain additional components that are unacceptably toxic to a subject to whom the formulation would be administered. The term “pharmaceutically acceptable carrier” refers to any carrier useful to solubilize and deliver an agent (for example the heterodimeric protein of the disclosure) to a subject. Many pharmaceutically acceptable carriers are known in the art. Merely by way of example, these include saline, phosphate buffer, or phosphate buffered saline. The composition may further routinely contain pharmaceutically acceptable concentrations of salt, buffering agents, preservatives, compatible carriers, supplementary immune potentiating agents such as adjuvants and cytokines and optionally other therapeutic agents. The composition may also include antioxidants and/or preservatives. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

### CLAUSES

1. A method for producing a heterodimeric protein that comprises two distinct IgG CH3 domains that are capable of forming a CH3-CH3 interface, said method comprising the steps of:
   providing:
      (a) a first protein comprising a first CH3 domain which comprises a positively charged amino acid residue at position 351 and 366, and
      (b) a second protein comprising a second CH3 domain which comprises a negatively charged amino acid residue at position 351 and 368,
      wherein the numbering is according to EU numbering,
   incubating the proteins of (a) and (b) together under reducing conditions to provide a reduced first protein and a reduced second protein; and
   reoxidising the reduced first and second proteins to obtain the heterodimeric protein.
2. The method of clause 1, wherein the first protein and/or the second protein comprises or is selected from the group consisting of: a monomeric protein, a homodimeric protein and a heterodimeric protein.
3. The method of any one of the preceding clauses, wherein the IgG CH3 domain is an IgG1, an IgG2, an IgG3, or an IgG4 CH3 domain, optionally wherein the CH3 domain is a human CH3 domain.
4. The method of any one of the preceding clauses, wherein the first protein and/or the second protein comprises or is selected from the group consisting of: an antibody and a half-antibody, or a fragment thereof, optionally wherein the antibody, half-antibody, or fragment thereof is a human antibody, half-antibody, or fragment thereof.
5. The method of any clause 4, wherein the fragment is a hinge-comprising monomeric Fc region or a hinge-comprising dimeric Fc region.
6. The method of clause 4 or 5, wherein the antibody, half antibody, or fragment thereof is an IgG1, an IgG2, an IgG3, or an IgG4 antibody, half antibody, or fragment thereof.
7. The method of any one of the preceding clauses, wherein the first protein is an antibody having a first binding specificity and the second protein is an antibody have a second distinct binding specificity.
8. The method of any one of the preceding clauses, wherein the first protein and/or the second protein is a homodimeric antibody.
9. The method of any one of the preceding clauses, wherein the first protein and/or the second protein is a heterodimeric antibody.
10. The method of any one of the preceding clauses, wherein the heterodimeric protein as obtained after reoxydising the reduced proteins is a heterodimeric antibody.
11. The method of clause 9 or 10, wherein the heterodimeric antibody is multivalent, optionally wherein the multivalent antibody is a bivalent, trivalent, tetravalent antibody or having up to six valences.
12. The method of any one of clauses 9 to 11, wherein the heterodimeric antibody is a multi-specific antibody, optionally wherein the multi-specific antibody is a bi-specific, tri-specific or quattro-specific antibody.
13. The method of any one of clauses 9 to 12, wherein the heterodimeric antibody comprises two non-identical light chains.
14. The method of any one of clauses 9 to 12, wherein the heterodimeric antibody comprises a single light chain.
15. The method of any one of clauses 9 to 12, wherein the heterodimeric antibody comprises three light chains.
16. The method of any one of clauses 9 to 12, wherein the heterodimeric antibody comprises three non-identical light chains.
17. The method of any one of clauses 9 to 12, wherein the heterodimeric antibody comprises two identical light chains and further light chain that is non-identical to the two identical light chains.
18. The method of any one of the preceding clauses, wherein the light chain is any member from the kappa or lambda families.
19. The method of any one of the preceding clauses, wherein the first protein and the second protein comprise an identical hinge region.
20. The method of any one of the preceding clauses, wherein In certain aspects, the first protein and the second protein comprise an IgG1 hinge region.
21. The method of any one of the preceding clauses, wherein the first protein and the second protein comprise an IgG2 hinge region.
22. The method of any one of the preceding clauses, wherein the first protein and the second protein comprise an IgG3 hinge region.
23. The method of any one of the preceding clauses, wherein the first protein and the second protein comprise an IgG4 hinge region.
24. The method of any one of the preceding clauses, wherein the first CH3 domain comprises: 351K and 366R, 351R and 366K, 351K and 366K, or 351R and 366R.
25. The method of any one of the preceding clauses, wherein the second CH3 domain comprises: 351D and 368E, 351E and 368D, 351D and 368D, or 351E and 368E.
26. The method of any one of the preceding clauses, wherein the first CH3 domain comprises 351K and 366K and the second CH3 domain comprises 351D and 368E.
27. The method of any one of the preceding clauses, wherein the first and/or second protein of a) and b) comprise one or more Fc modifications.
28. The method of any one of the preceding clauses, wherein the first and/or second protein of a) and b) are Fc engineered.
29. The method of any one of the preceding clauses, wherein the first and/or second protein of a) and b) are Fc silenced or Fc enhanced.
30. The method of any one of the preceding clauses, wherein the first and/or second protein of a) and b) comprise a CH2 domain having an ADCC-affecting mutation at position 235 and/or 236.
31. The method of any one of the preceding clauses, wherein the second protein is obtained independently from the first protein.
32. The method of any one of the preceding clauses, wherein the reducing conditions comprise:
(a) incubating the proteins in the presence of a reducing agent, optionally wherein the reducing agent comprises or is selected from the group consisting of: 2-mercaptoethylamine (2-MEA), dithiothreitol (DTT), dithioerythritol (DTE), glutathione, tris(2-carboxyethyl)phosphine (TCEP), L-cysteine, beta-mercapto-ethanol, thioglycolate, cysteamine, homocysteine, penicillamine and sodium borohydride; and/or
(b) incubating the proteins at a pH of between 6.0 and 12.0, optionally wherein the pH is between 7.0 and 8.0; and/or
(c) incubating the proteins at a redox potential of between −150 and −600 mV, optionally wherein the redox potential is between −250 and −400 mV.
33. The method of any one of the preceding clauses, wherein the method further comprises the step of enriching for and/or isolating the heterodimeric protein obtained after reoxidising the reduced proteins.
34. The method of clause 33, wherein the heterodimeric protein is enriched for and/or isolated using a method comprising or selected from the group consisting of: precipitation, centrifugation, filtration, size-exclusion chromatography, affinity chromatography, cation- and/or anion-exchange chromatography, and hydrophobic interaction chromatography.
35. An isolated heterodimeric protein obtainable by the methods according to any one of the preceding clauses.
36. An isolated heterodimeric antibody comprising a first IgG CH3 domain and a second IgG CH3 domain, wherein the first CH3 domain and the second CH3 domain are capable of forming a CH3-CH3 interface,
   wherein the first CH3 domain comprises one or more of the amino acid variants 366K, 366R, 351K or 351R, and
   wherein the second CH3 domain comprises one or more of the amino acid variants 351E, 351D, 368E or 368D,
   said heterodimeric antibody further comprising two or more light chains that have non-identical sequences.
37. The isolated heterodimeric antibody of clause 36, wherein the first CH3 domain comprises 351K and 366K and the second CH3 domain comprises 351D and 368E.
38. A pharmaceutical composition comprising an isolated heterodimeric protein according to clause 35, or an isolated heterodimeric antibody according to clause 36 or 37, and a pharmaceutically acceptable carrier.
39. The method of any one of clauses 1-34, wherein the first protein and/or the second protein comprises an antibody binding domain, a scFv, a ligand, protein receptor or cytokine.
40. The method of clause 39, wherein the heterodimeric protein is a bi- or multifunctional fusion protein.

### EXAMPLES

### Example 1: Transfection, Expression and Purification of IgG Antibodies for Use in Fab Arm Exchange (FAE)

The purpose of the following experiments was to directly compare the outcome of FAE using different variations listed in Table 1 (i.e. DEKK variations vs variations 405L-409R).

Expi293F™ cells (ThermoFisher Scientific) cultured in 100 mL Expi293F™ expression medium (ThermoFisher Scientific, cat. #A14351010), were transiently transfected with various expression vectors coding for Heavy Chains (HC) and Light Chains (LC), see Table 1), using ExpiFectamine™ 293 Transfection Kit (ThermoFisher Scientific, cat. #A14635) and OptiMEM I Reduced serum medium (Gibco, cat. #31985062).

TABLE 1
Expression vectors used to produce antibodies for FAE
Table 1: Expression vectors (#1-8) were used for transfections and
production of HC and LC molecules for eventual IgG production.

| | Half antibody | | | |
|---|---|---|---|---|
| | (HAb) as | CH3 | | |
| # | expressed | modification | HC sequence | LC sequence |
| 1 | HAb1-KK | KK | SEQ ID NO: 1 | SEQ ID NO: 9 |
| 2 | HAb2-KK | KK | SEQ ID NO: 2 | SEQ ID NO: 10 |
| 3 | HAb3-DE | DE | SEQ ID NO: 3 | SEQ ID NO: 11 |
| 4 | HAb4-DE | DE | SEQ ID NO: 4 | SEQ ID NO: 12 |
| 5 | HAb5-F405L | F405L | SEQ ID NO: 5 | SEQ ID NO: 9 |
| 6 | HAb6-F405L | F405L | SEQ ID NO: 6 | SEQ ID NO: 10 |
| 7 | HAb7-K409R | K409R | SEQ ID NO: 7 | SEQ ID NO: 11 |
| 8 | HAb8-K409R | K409R | SEQ ID NO: 8 | SEQ ID NO: 12 |

Expression vectors contain a DNA construct that code for a CH3 domain with the indicated modifications, a model heavy chain (HC) and a model light chain (LC).

Briefly, a DNA-Opti-MEM mixture and an Expifectamine-Opti-MEM mixture was prepared by diluting 50 μg plasmid DNA (0.1 mL from DNA stock with a concentration of 0.5 mg/mL) with 3 mL Opti-MEM™ I Medium and diluting 0.16 mL ExpiFectamine™ 293 Reagent with 2.8 mL Opti-MEM by swirl or inversion, followed by incubation for 5 minutes. The DNA-Opti-MEM mixture was then added to the Expifectamine-Opti-MEM mixture and the tube was inverted 4-5 times and incubated at room temperature for 15 minutes. Next, the entire volume of the ExpiFectamine™ 293/plasmid DNA complexes (about 6.06 mL) was added dropwise to said cells whilst gently swirling the flask during addition followed by incubation (37° C. incubator with >80% relative humidity and 8% CO₂ on an orbital shaker at 155 rpm).

18-22 hours post transfection, Enhancer 1 and Enhancer 2 of the ExpiFectamine™ 293 Transfection Kit were added to the transfection flask while gently swirling. Day 6 post transfection, medium was collected and cells were spun down at 500 g for 10 minutes at RT, supernatant was collected and transferred into a new 50 ml tube and cells were spun down at 3000 g for 20 minutes. Supernatants were filtered using a bottle top 0.45 μm filter and IgG concentrations were measured using the ForteBIO Octet-QK system, which is based on Bio-Layer Interferometry (BLI). This enabled real-time quantitation and kinetic characterization of biomolecular interactions. Supernatants were used for ÄKTA purification.

Volumes of culture supernatants that contained between 9-12 mg IgG were purified using AKTA pure system (Cytiva, EN490, serial number: 2031829) using protein A columns (GE Healthcare/cat #11-0034-95, according to GE Healthcare's instructions) and eluted in 0.1 M citrate buffer, pH 3.0 and immediately neutralized in an equal volume of 1.0 M Tris-HCL pH 8.0 or directly rebuffered to PBS using a desalting column (Cytiva #17-1408-01). Purified IgG molecules were used in FAE as described in Example 2.

The concentration was determined for all samples according to the Lambert-Beer law by measuring absorbance of the protein solution at 280 nm, corrected for PBS as blank, and adjusted for amino acid composition using the generic molar extinction coefficient of 1.45 mol⁻¹ dm³ cm⁻¹ for all. Antibody concentrations were all between 1.2 and 2.1 mg/mL and in PBS pH 7.4.

### Example 2: FAE Protocol

Briefly, IgG molecules were incubated at 31° C. at pH 7.4, in the presence of 75 mM MEA (p-Mercapto-ethylamine hydrochloride), without shaking. After 5 hours, samples were buffer exchanged to PBS pH 7.4 (Bex) at room temperature using Zeba plates. For reoxidation, samples were kept at 4° C. for at least 1 night.

The FAE reaction was performed in 96 well format on IgG molecules produced in Example 1 as, listed in Table 2.

TABLE 2
Expressed IgG antibodies used in FAE.
Table 2. Expressed IgG (half) antibodies
with indicated CH3 modifications.

| | Half | | Half | |
|---|---|---|---|---|
| FAE # | Antibody | CH3 | antibody | CH3 |
| 1 | HAb3-DE | DE | HAb1-KK | KK |
| 2 | HAb7-K409R | K409R | HAb5- | F405L |
| | | | F405L | |
| 3 | HAb4-DE | DE | HAb2-KK | KK |
| 4 | HAb8-K409R | K409R | HAb6- | F405L |
| | | | F405L | |
| 5 | HAb3-DE | DE | | |
| 6 | HAb1-KK | KK | | |
| 7 | HAb7-K409R | K409R | | |
| 8 | HAb5-F405L | F405L | | |
| 9 | HAb4-DE | DE | | |
| 10 | HAb2-KK | KK | | |
| 11 | HAb8-K409R | K409R | | |
| 12 | HAb6-F405L | F405L | | |

FAE reactions# 1-4 include a mixture of two IgG molecules while FAE reactions # 5-12 include individual IgG as controls.

Fresh MEA stock solution (750 mM) was prepared by dissolving 852 mg Cysteamine hydrochloride (Sigma cat #30078) in 5-6 mL PBS (pH7.4). pH was adjusted to pH 7.3-7.5 by adding NaOH (5M) to the solution at room temperature and the solution was filled with PBS pH 7.4 up till the final volume of 10 mL to achieve 750 mM stock solution. The solution was filtered through 0.2 μm filter before use.

Briefly, IgG samples listed in Table 2 were prepared in a deep well plate (plate 1, 175 μL final volume adjusted by adding PBS pH 7.4) using IgG at a concentration of 1.1 mg/mL. In 12 wells of another deep well plate (plate 2), 11 μL of reducing agent 750 mM MEA was pipetted. 100 μL of each prepared sample of plate 1 was gently mixed with the MEA in plate 2, which was covered with an aluminum seal and kept at 31° C. for 5 hours without shaking. Material remaining in plate 1 (75 μL) was used as non-reacted controls i.e. not exposed to MEA or Bex and stored at 4° C. in the dark until further use. After incubation, 100 μL of each sample was used for buffer exchange (bex) with the Zeba™ Spin Desalting plates, 96-well (Thermo Fisher Scientific, cat. #89807).

### Buffer Exchange:

Samples were buffer exchanged against PBS pH 7.4 (1×, Gibco cat. #10010-015) using Zeba™ Spin Desalting kit (Thermo Fisher Scientific, cat. #89807) to remove the reduction agent and allow reformation of disulphide bonds.

Briefly, Zeba™ spin desalting plates were equilibrated to room-temperature and assembled on top of the wash plate. The plate assembly was centrifuged to remove storage solution and the wash plate was blotted dry on paper towel. Three washing steps were performed by addition of washing buffer (1×, Gibco cat. #10010-015), after which plates were centrifuged, flow through was discarded and dry blotted on towel paper.

Next, Zeba™ desalting plates were stacked on top of the collection plate and IgG samples were loaded into the wells and centrifuged. The flow through containing IgG was transferred into another Zeba™ plate and centrifuged. To achieve a more thorough buffer exchange, the flow through was transferred again into another Zeba™ plate and centrifuged. All centrifugations were performed at 1000×g for 2 minutes. The third flow through that contains the sample which has passed three times through the Zeba™ plate, was retained for IgG concentration measurements. Buffer-exchanged samples were kept in the collector plate covered and kept at 4° C. overnight (without further pipetting, shaking or mixing) to allow material to fully re-oxidize. Buffer exchanged samples and the non-reacted controls kept on plate 1 at 4° C. were measured on Lunatic system as mentioned in Example 1. IgG molecules as obtained were used in SDS-PAGE using Labchip under reducing and non-reducing conditions where indicated and as appropriate using CIEX analysis and HP-SEC.

### Example 3: LabChip Analysis

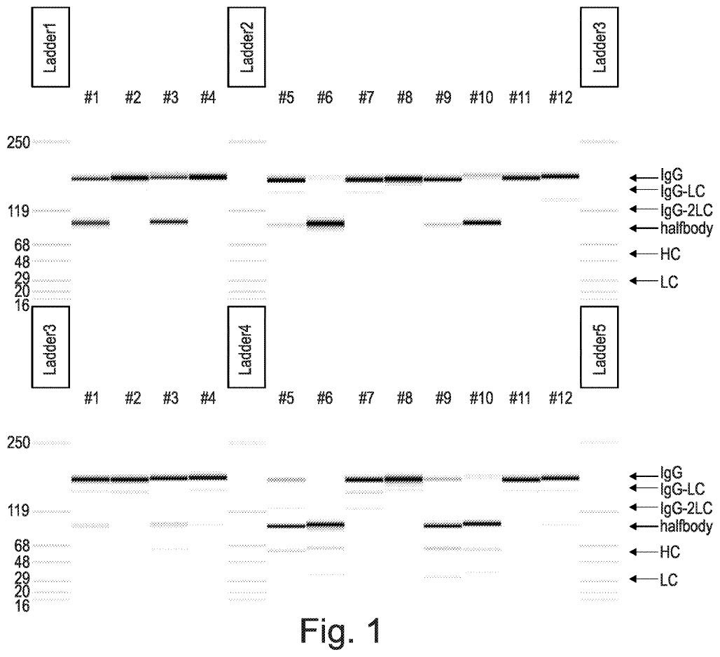

Approximately 1 μg IgG of the purified reaction products, including non-reacted controls were analyzed by Labchip (LabChip GXII Touch HT; Perkin Elmer) under non-reducing conditions using Protein Clear HR Reagent kit (Dye Solution, Sample Buffer, Protein Gel Matrix, Protein Ladder, Lower Marker, Wash Buffer; Perkin Elmer CLS960014) and Protein Express Assay LabChip for use with GXII Touch HT (Elmer 760499) according to the manufacturer's instructions. From samples run under non-reduced conditions, the IgG, half body, and IgG-1 LC bands were quantified using Labchip RX reviewer software. Only samples where the IgG-1 LC band made up less than 15% signal of samples subjected to FAE were analyzed. Also, care was taken that only samples of which the starting material contained more than 95% intact molecules (IgG dimer or half body) were included. Results are given in FIG. 1.

Samples taken from before the FAE reaction behaved as follows: Species containing CH3-DE variations mainly formed DEDE homodimers, while samples containing CH3 KK variations mainly formed half-bodies. Single arm productions with CH3 variations 405L or 409R predominantly formed homodimers. Samples with mixtures of DE/KK or 405L/409R showed bands as set out in FIG. 1; samples from reactions containing DE/KK CH3 variations showed mixtures of homodimer and half-bodies, while samples from proteins containing CH3 variations 405L/409R showed predominantly homodimers.

Single productions from samples obtained after FAE reactions with DE or KK CH3 variations showed predominantly half-bodies, while samples with 405L or 409R CH3 variations form homodimers.

Samples with mixtures of CH3-containing DE/KK species obtained after FAE reactions showed an IgG peak with half-body contaminants, while an IgG peak with unseparated homodimer contaminants was detected in the samples containing 405L/409R species.

### Example 4: HP-CIEX Analysis

To confirm that FAE reaction indeed led to generation of bispecific antibodies, all samples generated in Example 2 (reaction products and non-reacted controls) were analysed by CIEX (Agilent 1260 series). The column (TSK gel SP-STAT 7 μm, 4.6 mm ID×10 cm L; Tosoh cat #21964) was equilibrated using phosphate buffer (Buffer A) with a low ionic strength (25 mM Sodium Phosphate pH 6.0±0.05, composed of Sodium Phosphate monobasic, dihydrate (NaH₂PO₄, 2 H₂O; Sigma, ref. 71500) and Sodium Phosphate dibasic, dihydrate (Na₂HPO₄, 2H₂O; Sigma, ref. 71643) dissolved in Milli-Q water, filtered by 0.45 μm membrane filter. The antibodies were then displaced from the column by increasing the Na+ concentration by running a gradient of Phosphate buffer with an increasing percentage of Phosphate buffer with salt (Buffer B) (25 mM Sodium Phosphate, 1M NaCl pH 6.0, composed of Sodium Phosphate monobasic, dihydrate (NaH₂PO₄, 2 H₂O; Sigma, ref. 71500) and Sodium Phosphate dibasic, dihydrate (Na₂HPO₄, 2H₂O; Sigma, ref. 71643) and NaCl (Sigma, ref. S3014) dissolved in Milli-Q water, filtered by 0.45 μm membrane filter). The injected sample mass for all test samples and controls was 10 μg in injection volumes between 10 and 100 μL. The chromatograms were analysed for the peak patterns and retention times. The peak areas for the major peaks were observed based on the 220 nm results.

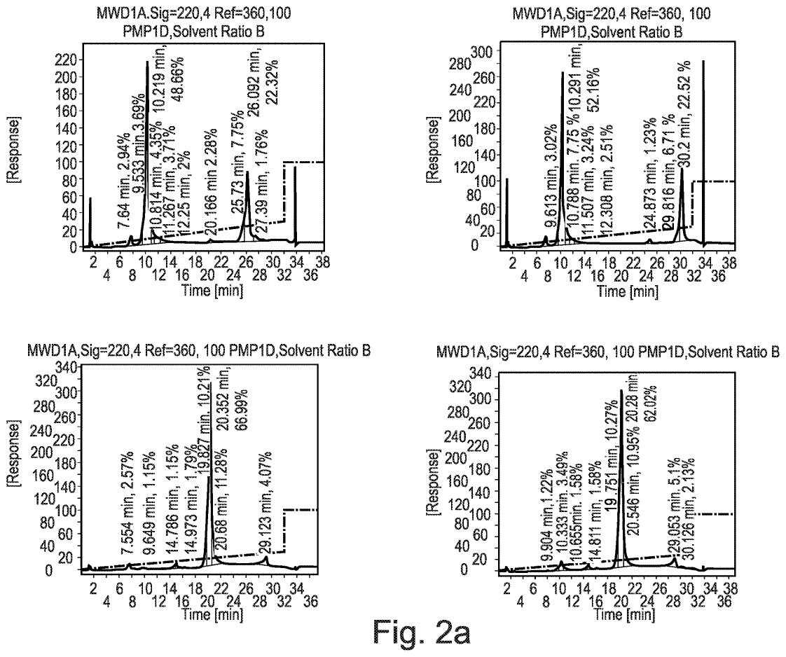

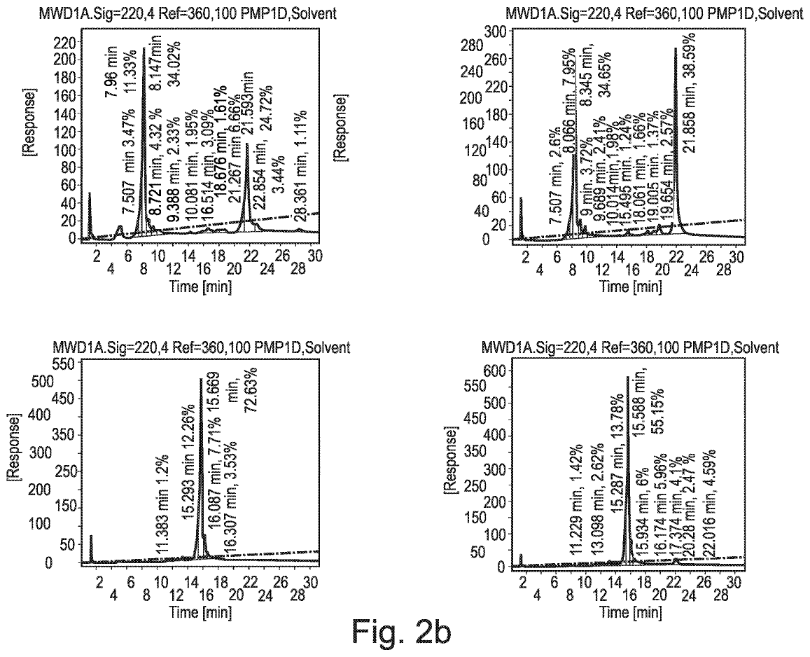

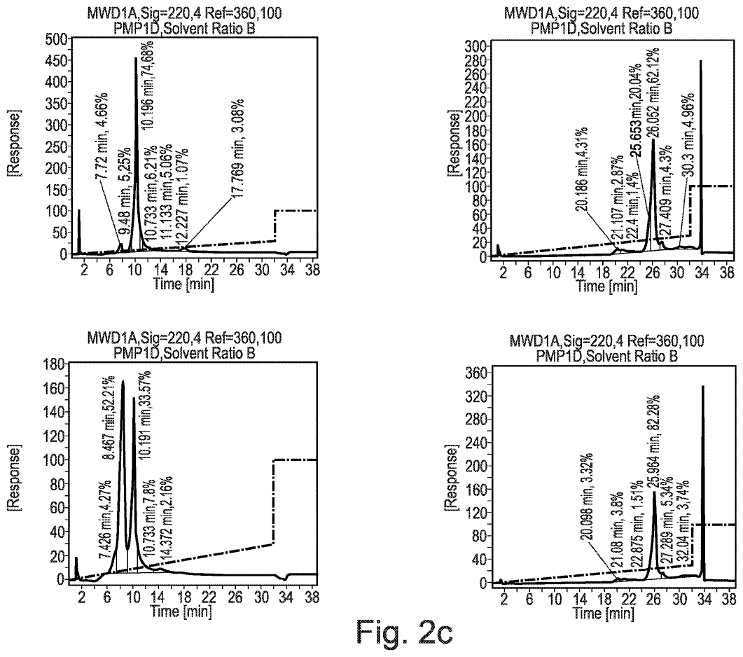

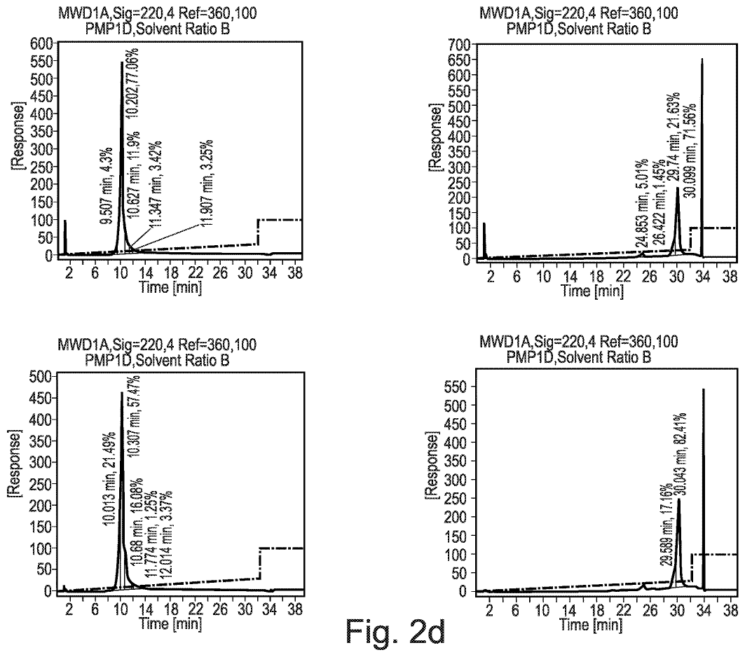

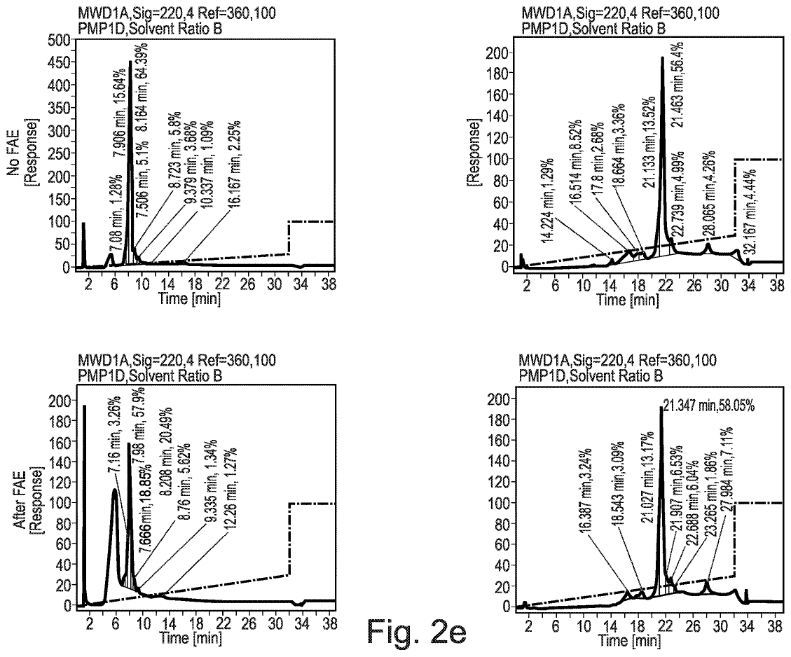

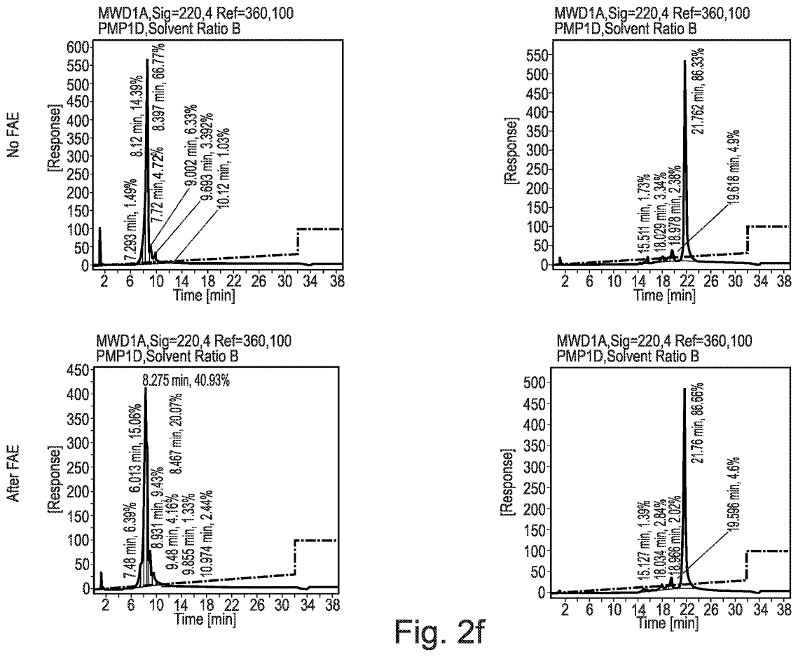

The percentage of bispecific antibodies in samples that underwent FAE were used to calculate efficiency as a percentage of FAE reactions. Differences between samples that underwent FAE and non-reacted controls were used to identify bispecific antibodies and contaminants after FAE. Results are given in FIGS. 2a-f.

FAE using DEKK variations led to only about 1% homodimers for reaction #1 and no detectable homodimers for reaction #3, while FAE using 405L/409R gave rise to about 3.5% homodimers for reaction #2 and about 4.6% for reaction #4 (see FIGS. 2a/b).

Also, samples taken from reactions 1 # and #3 after FAE, showed a peak appeared (at about 21 mins, and about 16, resp.) which was absent in the individual samples and which made up about 88% or 96%, respectively, of the material eluting from CIEX that corresponds to the DEKK heterodimer. After FAE, in samples obtained from FAE reactions #5 and #9, an early eluting peak appeared (at about 8.5 mins and 1-6 minutes, respectively) which indicated formation of DE half body. Samples taken from reactions #2 and #4 after FAE, a peak that represents heterodimers (around 16 minutes) appeared that made up about 83% or 81%, respectively, which was absent in individual samples of the material that eluted from CIEX that corresponds to the 405L/409R heterodimer.

### Example 5: HP-SEC Analysis

To detect aggregates and the contents of IgG dimers and half antibodies in samples before and after FAE under native conditions, samples generated according to Example 2, (i.e. reaction products and non-reacted controls) were analysed by HP-SEC (Agilent 1260 series) using TSK-gel G3000SWxl (Tosoh Bioscience—808541), TSK guard column SWXL (Tosoh Bioscience—808543), HP-SEC buffer (200 mM Sodium Phosphate, 50 mM NaCl, pH7.0; composed of Sodium Phosphate monobasic, dihydrate (NaH2PO4, 2H2O; Sigma, ref. 71500), Sodium Phosphate dibasic, dihydrate (Na2HPO4, 2H2O; Sigma, ref. 71643), and NaCl (Sigma, ref. S3014) dissolved in Milli-Q water), filtered by 0.45 μm membrane filter.

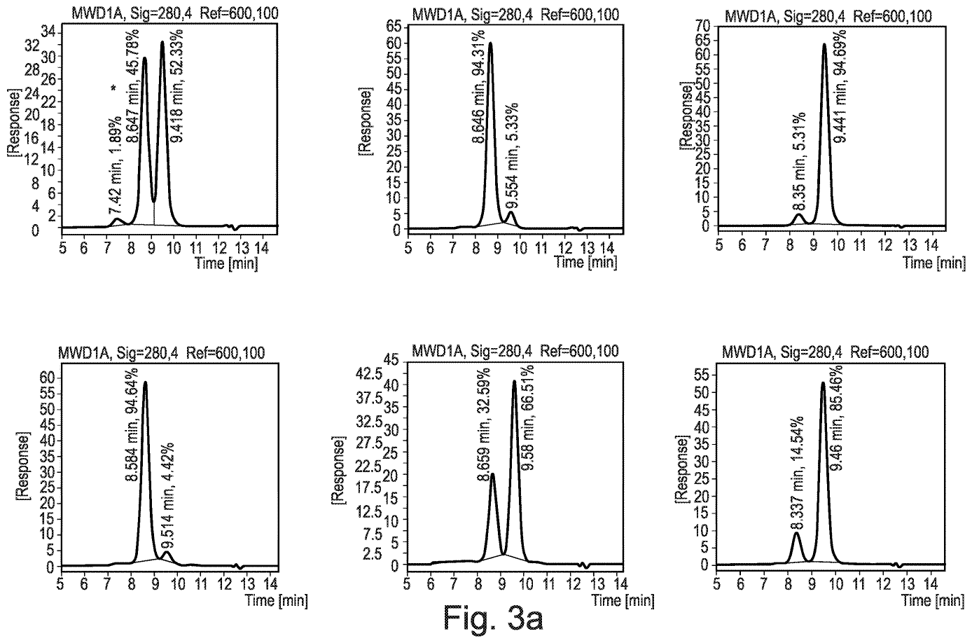

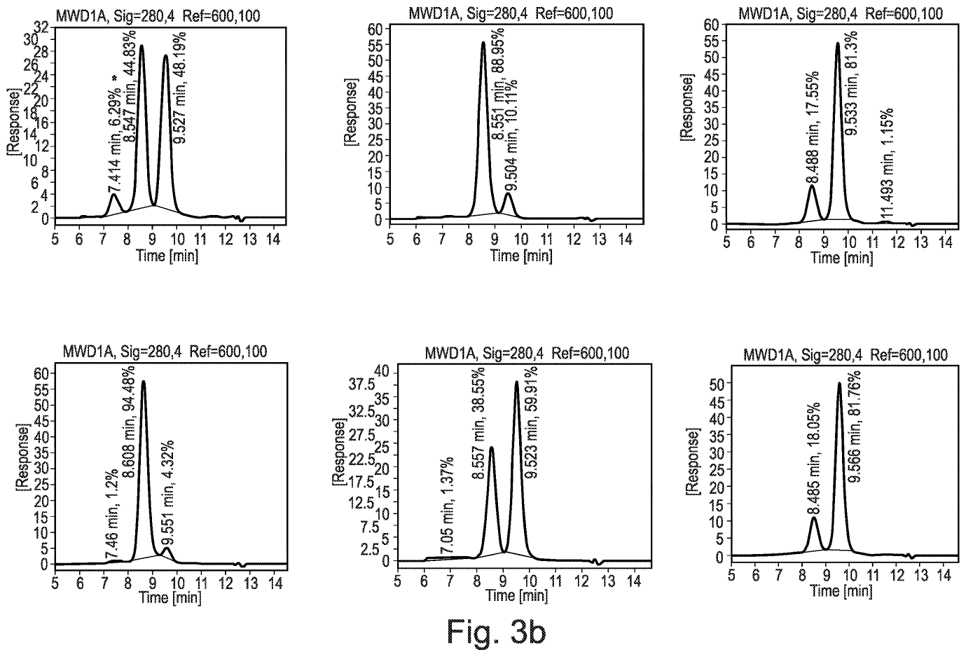

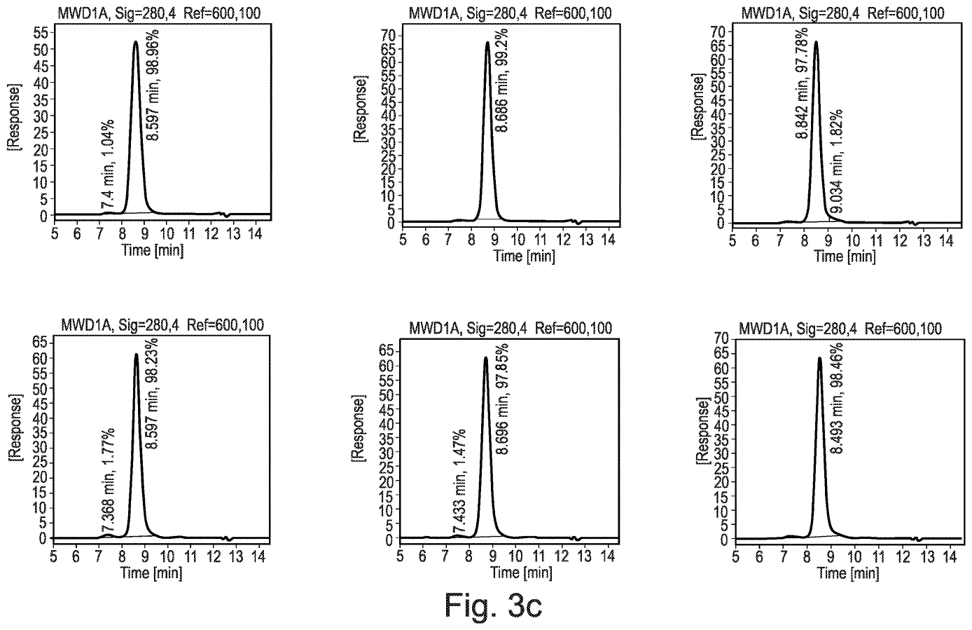

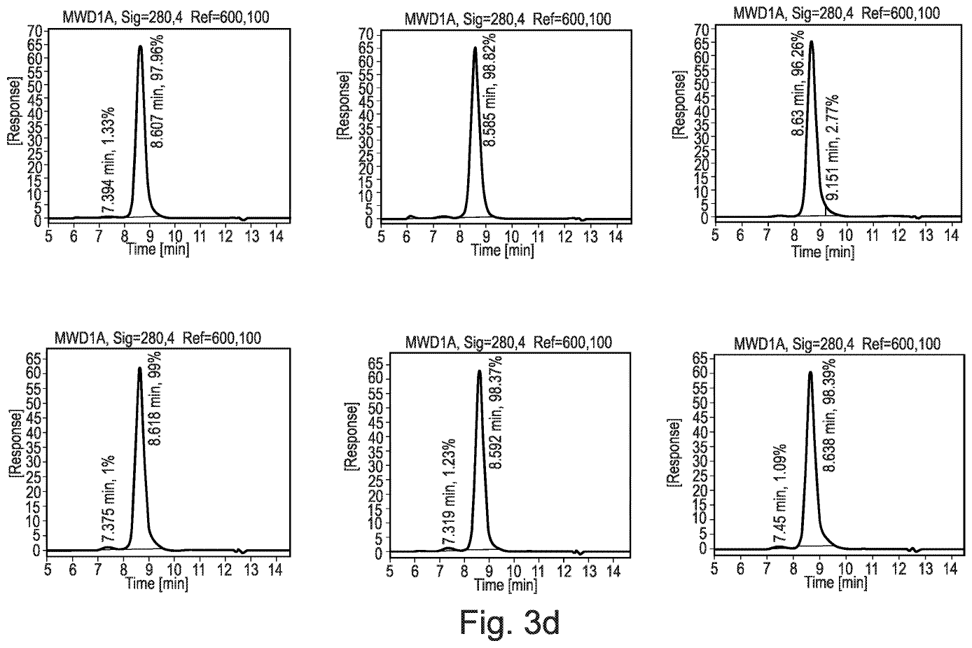

The same amount of samples was injected (20 μg of each sample at injection volumes between 10 and 100 μL). Data as obtained was used to estimate the purity (% intact IgG dimer or half body) in samples. To ensure quality of the samples is sufficient, any starting material should contain more than 95% intact molecules. Else, additional polishing or repeated production was considered. The chromatograms were analysed for the retention times and relative peak areas for the peaks observed based on the 280 nm results. Results are shown in FIG. 3. The main peak presents IgG-IgG dimer since there are no aggregates in the same samples if they are analysed individually.

After FAE, reactions containing CH3 variations with DE and KK (i.e. #1 and #3), a main peak that corresponds in size to an IgG was detected without aggregates but with small amounts of half body. After FAE, reactions containing CH3 variations with 405L and 409R (i.e. #2 and #4), a main peak that corresponds in size to an IgG was detected without aggregates. After FAE of individual single productions from samples obtained after FAE reactions with DE (#5 and #9) or KK (#6 and #10) CH3 variants showed predominantly halfbodies, while samples with 405L (#8 and #12) or 409R (#7 and #11) CH3 variants form homodimers.

### Example 6: Upscaled FAE Protocol

Briefly, IgG molecules were incubated in the presence of p-Mercapto-ethylamine hydrochloride without shaking. Next, samples were buffer exchanged (Bex) to PBS using the AKTA Pure 25 system equipped with a Spark ALIAS autosampler and a single HiPrep 26/10 desalting column, Cytiva. For reoxidation, samples were kept at 4° C. for a sufficient time to allow full reoxidation to occur. The FAE reaction was performed on IgG molecules produced in Example 1 as, listed in Table 3.

TABLE 3
Expressed IgG antibodies used in FAE.

| # | Half antibody A | Half antibody B | Ratio HAb-A:HAb-B |
|---|---|---|---|
| 1 | HAb3-DE | HAb1-KK | 1.25:1 |
| 2 | HAb3-DE | HAb1-KK | 1:1.25 |
| 3 | HAb7-K409R | HAb5-F405L | 1.25:1 |
| 4 | HAb7-K409R | HAb5-F405L | 1:1.25 |
| 5 | HAb4-DE | HAb2-KK | 1.25:1 |
| 6 | HAb4-DE | HAb2-KK | 1:1.25 |
| 7 | HAb8-K409R | HAb6-F405L | 1.25:1 |
| 8 | HAb8-K409R | HAb6-F405L | 1:1.25 |

Fresh MEA stock solution (750 mM) was prepared by dissolving 852 mg cysteamine hydrochloride (Sigma cat #30078) in 5-6 mL PBS (pH7.4). pH was adjusted to pH 7.3-7.5 by adding NaOH (5M) to the solution at room temperature and the solution was filled with PBS pH 7.4 to a final volume of 10 mL to obtain the 750 mM stock solution and was filtered using a 0.2 μm filter before use.

IgG mixtures listed in Table 3 were prepared at 1.1 mg/mL final concentration, 3.15 mL final volume adjusted by adding PBS (diluted from 10× stock Gibco Cat #70011-051 pH 7.4 using Fresenius Versylene sterile endotoxin-free water, Cat #B230531)). 100 μL of this mixture was withdrawn and kept at 4 degree as non-reacted control i.e. not exposed to MEA or Bex and stored at 4° C. in the dark until further use. To start the FAE, to the remaining 3.05 mL, 350 μL of 75 mM MEA was added and solutions were mixed gently and kept at 31° C. for 5 hours without shaking.

### Buffer Exchange:

After 5 hours, samples were buffer exchanged (Bex) to PBS (diluted from 10× stock Gibco Cat #70011-051 pH 7.4 using Fresenius Versylene sterile endotoxin-free water, Cat #B230531) using AKTA Pure 25 system equipped with a Spark ALIAS autosampler and one HiPrep 26/10 desalting column, Cytiva Cat #17-5087-01, at 6 mL/min flow rate and 20 degree celsius. For reoxidation, samples were kept at 4° C. for 64 hours (without further pipetting, shaking or mixing) to allow material to fully re-oxidize. For quality control purposes, 60-100 μg of each sample was kept before gel filtration purification took place (i.e. a pre-GF control).

### Gel Filtration

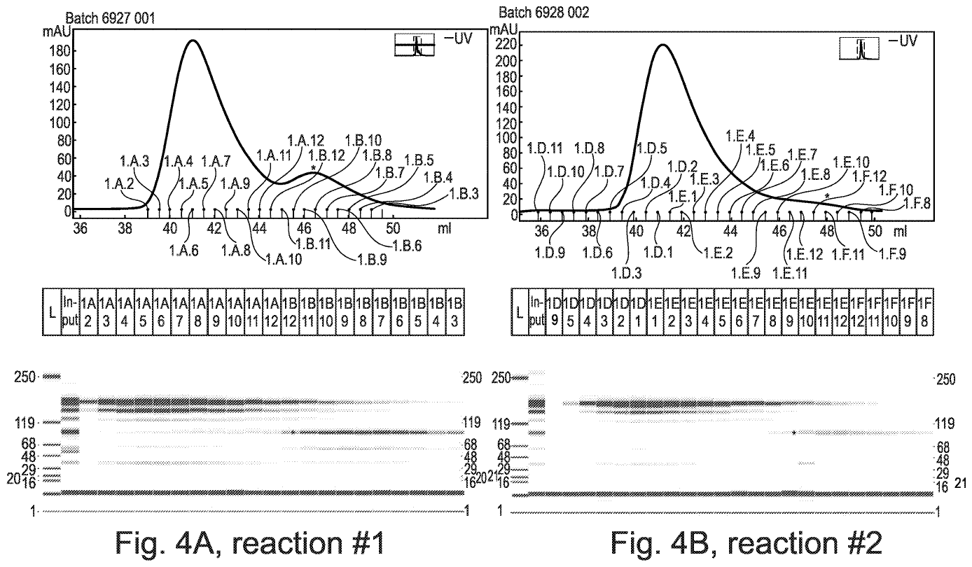

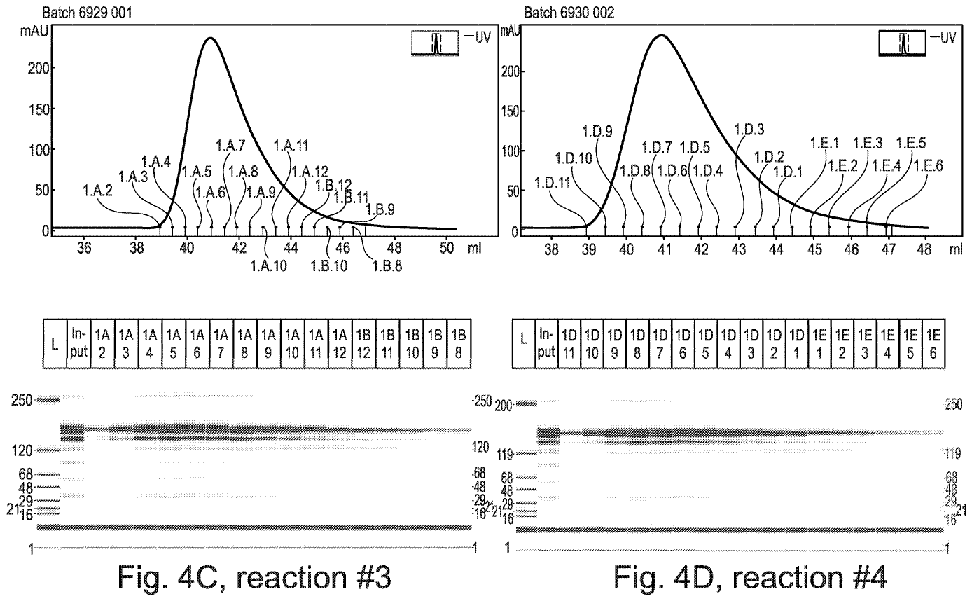

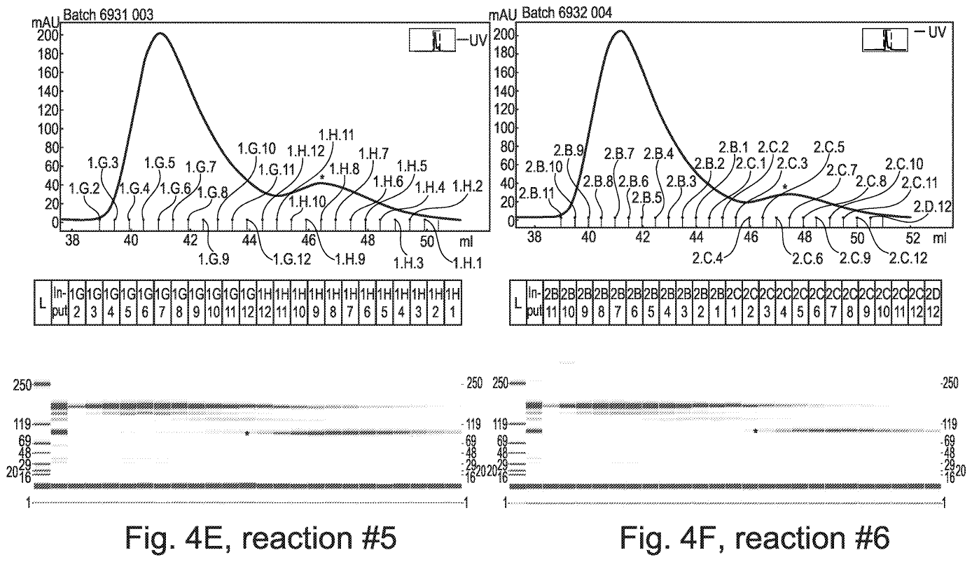

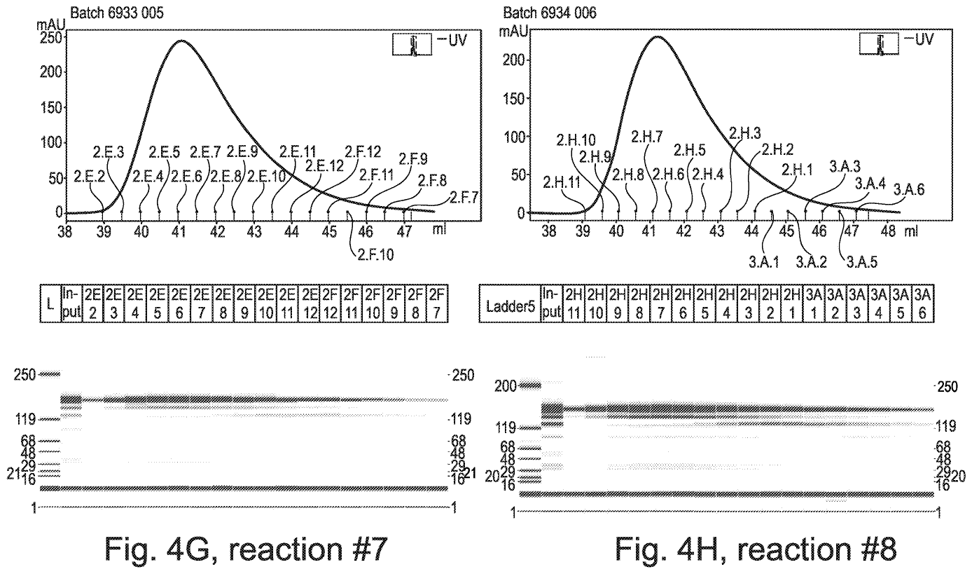

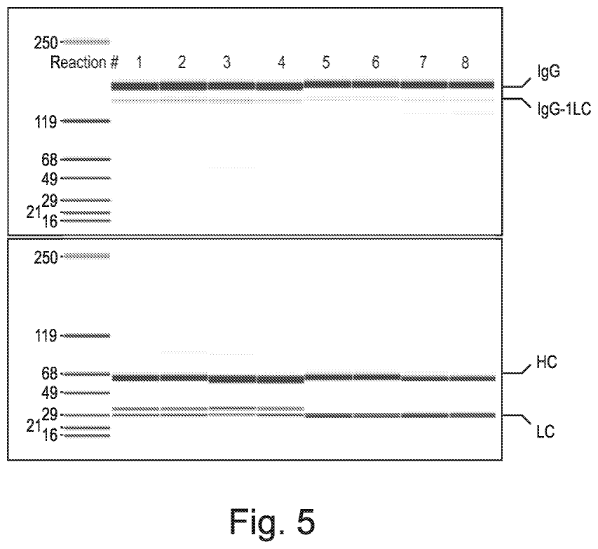

Buffer exchanged samples were concentrated with an Amicon 15 Ultra (30 kDa molecular weight cutoff) device (Merck/Millipore Cat #UFC903096) to a volume of 2.4+/−0.4 mL (~1.5+/−0.25 mg/mL protein concentration). Then the material was loaded on an AKTA Pure 25 system equipped with a Spark ALIAS autosampler using PBS as mobile phase (PBS prepared as above) for Gel filtration purification to separate samples based on size with a Superdex 200 increase 16/40 column, Cytiva Cat #29321905, at a flow rate of 1 mL/min (fraction size 0.5 mL, autosampler injection loop 10 mL, temperature 20° C.). Detection of samples was by UV light according to the manufacturer's instructions. Results of reactions #1-8 from Table 3 are shown in FIG. 4a-4h, upper panels.

### LabChip Analysis

Fractions from the gel filtration column were analyzed on LabChip GXII Touch instrument under non-reducing conditions to visualize the possible presence of IgG dimers and half bodies in gel filtration fractions as obtained. Labchip analysis was done with the HT Protein Express chip (Perkin-Elmer Cat #760499), Protein Clear HR reagent kit (Perkin Elmer CL8960014). Conditions for Labchip analysis: input samples and fractions with a concentration of more than 1 mg/mL were diluted to 1 mg/mL using PBS. 1 μL sample was mixed with 7 μL non-reducing sample buffer (which contains 9 mM N-Ethylmaleimide) in a PCR plate and incubated 10 min at 70° C. Subsequently, 14 μl H₂O was added, after which the plate was centrifuged for 2 mins at 2800×g. For sample analysis, a LabChip GXII Touch instrument was used according to the manufacturer's instructions using the standard “HT Protein Express” script. Results of reactions #1-8 from Table 3 are shown in FIG. 4a-4h, lower panels. Samples taken after FAE contain mainly IgG dimers. Nearly no aggregates are observed. An asterisk for samples of #1, #2, #5, #6 indicates the IgG half body peaks on Gel filtration and Labchip analysis which are separated from the IgG dimer.

Fractions which contained IgG dimer but no half body were pooled and protein concentration of pooled samples was determined on a Little Lunatic UV/Vis spectrophotometer, according to the manufacturer's instructions (Unchained Labs). Absorbance at 280 nm was measured using the total protein program, with water as blank and an extinction coefficient of 1.45 mL/mg/cm, according to the manufacturer's instructions.

Pooled samples derived from reactions #1-8 from Table 3 were again analyzed using Labchip under non-reducing conditions as described above as well as under reducing conditions using sample buffer which contained 35 mM DL-Dithiothreitol (Sigma cat #43819) are shown in FIG. (non-reduced: upper panel, reduced: lower panel). The results from non-reduced conditions indicate that the purity of all IgG antibodies is at least 98% and that a very minor amount of an IgG with a single LC is seen. Hence, FAE did not cause a significant loss of light chain using these samples. The results from the reduced conditions indicate that bispecific antibodies from reactions #1-4 and #5-8 share the same mixture of light chains which indicates the presence of two different Fab arms in the IgG samples.

Also, the “pre-GF control samples” of reactions #1-8 taken before and after FAE, were analysed by reducing and non-reducing LabChip analysis according to the procedure given above. For all reactions, samples were verified to contain halfbodies prior to subjecting the samples to FAE while after FAE samples showed less halfbodies (data not included).

### Example 7: HP-CIEX Analysis

To confirm that FAE reaction indeed led to generation of bispecific antibodies, all samples generated in Example 2 (reaction products and non-reacted controls) were analysed by CIEX (Agilent 1260 series). The column (TSK gel SP-STAT 7 μm, 4.6 mm ID×10 cm L; Tosoh cat #21964) was equilibrated using phosphate buffer (Buffer A) with a low ionic strength (25 mM Sodium Phosphate pH 6.0±0.05, composed of Sodium Phosphate monobasic, dihydrate (NaH₂PO₄, 2 H₂O; Sigma, ref. 71500) and Sodium Phosphate dibasic, dihydrate (Na₂HPO₄, 2H₂O; Sigma, ref. 71643) dissolved in Milli-Q water, filtered by 0.45 μm membrane filter. The antibodies were then displaced from the column by increasing the Na+ concentration by running a gradient of Phosphate buffer with increasing salt percentage (Buffer B) (25 mM Sodium Phosphate, 1M NaCl pH 6.0, composed of Sodium Phosphate monobasic, dihydrate (NaH₂PO₄, 2 H₂O; Sigma, ref. 71500) and Sodium Phosphate dibasic, dihydrate (Na₂HPO₄, 2H₂O; Sigma, ref. 71643) and NaCl (Sigma, ref. S3014) dissolved in Milli-Q water, filtered by 0.45 μm membrane filter). The injected sample mass for all test samples and controls was 10 μg with injection volumes between and 100 μL. The chromatograms were analysed for the peak patterns, retention times and peak areas for the major peaks observed based on the 220 nm results.

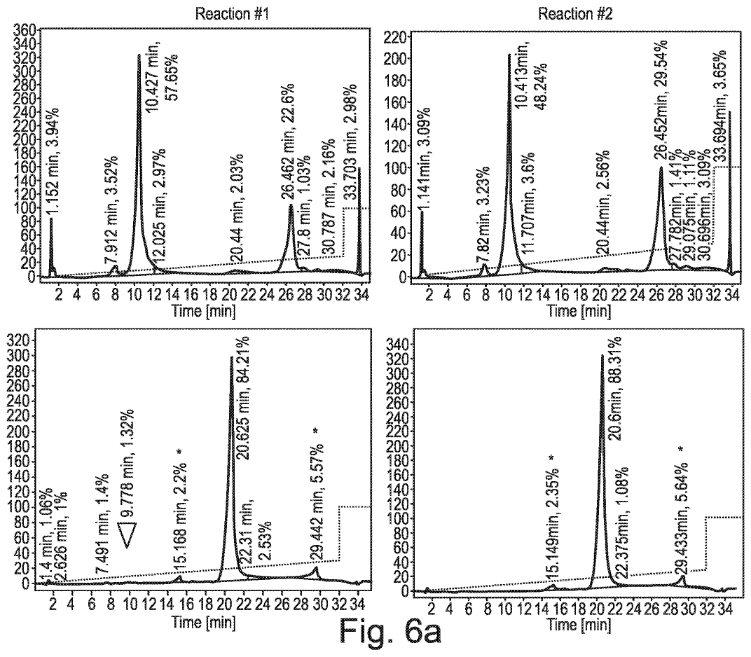

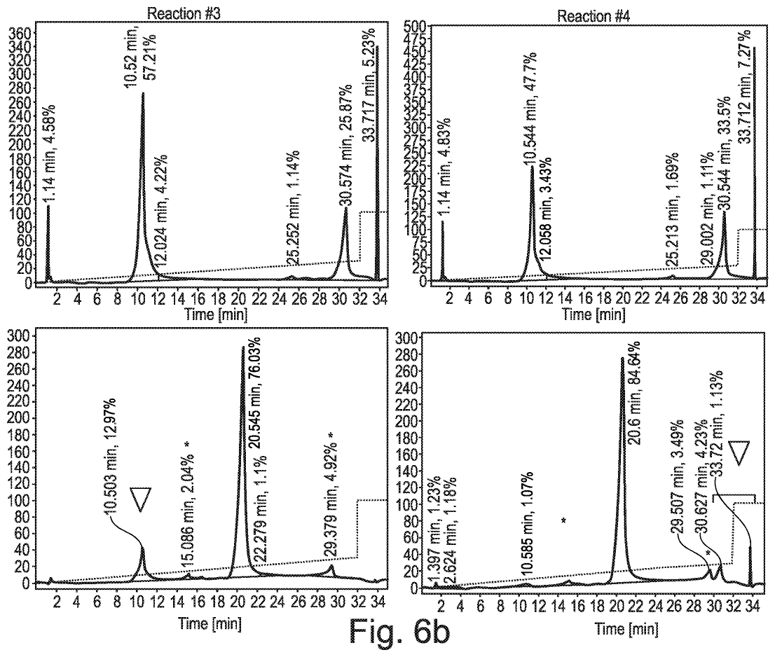

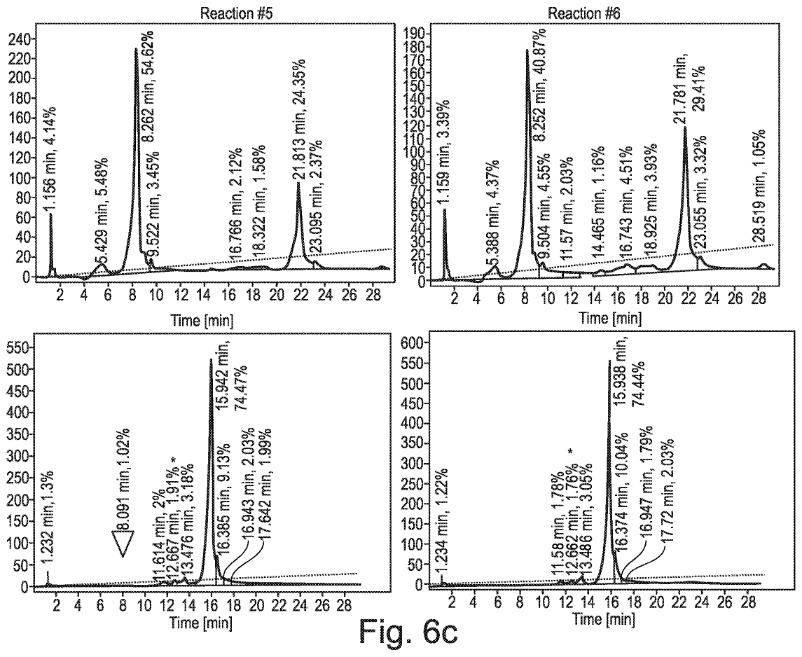

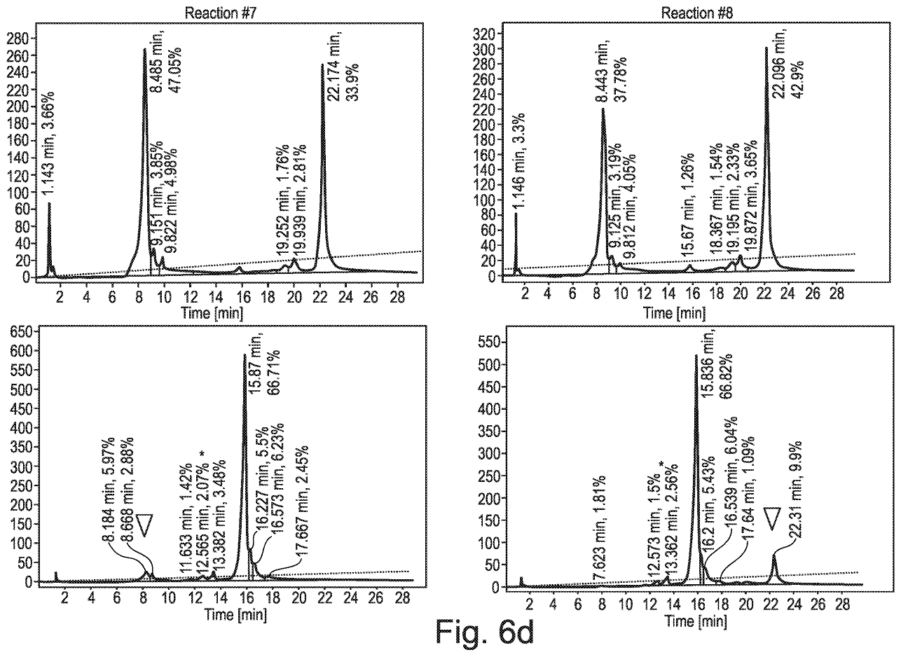

The results in the respective lower panels of FIG. 6 show that FAE using DE/KK variations led to only about 1% homodimers (see reactions #1 and #5) or even undetectable amounts of homodimers (see reactions #2 and #6). FAE using variations 405L/409R gave rise to about 5-13% homodimers (see reactions #3, #4, #7 and #8).

The results shown in the lower panels of FIG. 6a and FIG. 6c show peaks appearing which were absent before FAE which made up about 87-89% or 95-96% of the material eluting from CIEX that corresponds to the DEKK heterodimer (i.e. a peak at about 21 mins for #1 and #2 and at about 17 minutes for reactions #5 and #6).

The results shown in the lower panels of FIG. 6b and FIG. 6d show peaks appearing which were absent before FAE which made up about 77-84% or 83-88%, respectively, of the material eluting from CIEX that corresponds to the 405L/409R heterodimer (i.e. a peak at about 21 minutes for reactions #3 and #4 and at about 16 mins for #7 and #8). The asterisks in FIG. 6a-d show peaks that are detected in each sample from #1 to #8 indicating these occur independently from either the DE/KK or 405L/409R Fc format used. Based on the position relative to the main heterodimer peak, it is expected these can be removed in a straightforward manner using standard separation techniques.

Taken together, the results show that using either the DE/KK or 405L/409R substitutions, heterodimers could be produced at larger scale than shown in Examples 1-5 and with non-equal ratios of starting material. Gel filtration-purified samples carrying 405L/409R substitutions contain 5-13% unwanted homodimers while in samples carrying the DE/KK substitutions, contaminants were more successfully removed with a reported maximum level of 1% homodimers. Using the DEKK-based substitutions, a higher purity was obtained compared to the 405L/409R substitutions (87-96% vs 77-88%, respectively).

Also, estimated overall recovery of bispecific antibodies in terms of milligram protein after FAE reactions is comparable between the two substitution systems, but samples wherein FAE was performed using DE/KK substitutions were reported to have higher purity.

Of note, purified bispecific antibodies, generated by FAE, were confirmed to still bind to their cognate antigens by ELISA. Binding to other targets was shown to not occur. All antibodies prepared by FAE show specific binding to their targets leading to the conclusion that antibody specificity is maintained after FAE despite the reduction and re-oxidation of the Fab arms. Also, irrespective of the DE:KK ratios selected before FAE, bispecific antibodies were found to bind similarly to each antigen.

SEQUENCES

SEQ ID NO. 1
EVQLVQSGAEVKKPGASVKVSCKASGFTFTSYYIHWVRQAPGQGLEWIGWIYPENDNTKYNE
KFKDRVTITADTSTSTAYLELSSLRSEDTAVYYCARDGYSRYYFDYWGQGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTKPPSREEMTKNQVSLKCLVKGFYPS
DIAVEWESNGQPENNYKTTPPVLDSDGSFFLYSKLTVDKSRWQQGNVFSCSVMHEALHNHYT
QKSLSLSPGK
SEQ ID NO. 2
QVQLVESGGGVVQPGGSLRLSCAASGFTFSNAWMHWVRQAPGKGLEWVAQIKDKSQNYAT
YVAESVKGRFTISRADSKNSIYLQMNSLKTEDTAVYYCRYVHYAAGYGVDIWGQGTTVTVSSA
STKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTKPPSREEMTKNQVSLKCLVKG
FYPSDIAVEWESNGQPENNYKTTPPVLDSDGSFFLYSKLTVDKSRWQQGNVFSCSVMHEALH
NHYTQKSLSLSPGK
SEQ ID NO. 3
EVQLLEPGGGLVQPGGSLRLSCEASGSTFSTYAMSWVRQAPGKGLEWVSGFSGSGGFTFYA
DSVRGRFTISRDSSKNTLFLQMSSLRAEDTAVYYCAIPARGYNYGSFQHWGQGTLVTVSSAST
KGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTDPPSREEMTKNQVSLTCEVKGFY
PSDIAVEWESNGQPENNYKTTPPVLDSDGSFFLYSKLTVDKSRWQQGNVFSCSVMHEALHNH
YTQKSLSLSPGK
SEQ ID NO. 4
EVQLVQSGAEVKKPGESLKISCKGSGYSFTSYWIGWVRQMPGKGLEWMGIIFPDDSDTRYSP
SFQGQVTISADKSISTAYLQWSSLKPSDTAMYYCVRLGGYSGYAEDFVDFWGQGTLVTVSSAS
TKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTDPPSREEMTKNQVSLTCEVKGF
YPSDIAVEWESNGQPENNYKTTPPVLDSDGSFFLYSKLTVDKSRWQQGNVFSCSVMHEALHN
HYTQKSLSLSPGK
SEQ ID NO. 5
EVQLVQSGAEVKKPGASVKVSCKASGFTFTSYYIHWVRQAPGQGLEWIGWIYPENDNTKYNE
KFKDRVTITADTSTSTAYLELSSLRSEDTAVYYCARDGYSRYYFDYWGQGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCLVKGFYPS
DIAVEWESNGQPENNYKTTPPVLDSDGSFLLYSKLTVDKSRWQQGNVFSCSVMHEALHNHYT
QKSLSLSPGK
SEQ ID NO. 6
QVQLVESGGGVVQPGGSLRLSCAASGFTFSNAWMHWVRQAPGKGLEWVAQIKDKSQNYAT
YVAESVKGRFTISRADSKNSIYLQMNSLKTEDTAVYYCRYVHYAAGYGVDIWGQGTTVTVSSA
STKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFLLYSKLTVDKSRWQQGNVFSCSVMHEALH
NHYTQKSLSLSPGK
SEQ ID NO. 7
EVQLLEPGGGLVQPGGSLRLSCEASGSTFSTYAMSWVRQAPGKGLEWVSGFSGSGGFTFYA
DSVRGRFTISRDSSKNTLFLQMSSLRAEDTAVYYCAIPARGYNYGSFQHWGQGTLVTVSSAST
KGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLYSRLTVDKSRWQQGNVFSCSVMHEALHNH
YTQKSLSLSPGK
SEQ ID NO. 8
EVQLVQSGAEVKKPGESLKISCKGSGYSFTSYWIGWVRQMPGKGLEWMGIIFPDDSDTRYSP
SFQGQVTISADKSISTAYLQWSSLKPSDTAMYYCVRLGGYSGYAEDFVDFWGQGTLVTVSSAS
TKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKGF
YPSDIAVEWESNGQPENNYKTTPPVLDSDGSFFLYSRLTVDKSRWQQGNVFSCSVMHEALHN
HYTQKSLSLSPGK
SEQ ID NO. 9
DIVMTQSPDSLAVSLGERATINCKSSQSLLNSRTRKNYLAWYQQKPGQSPKLLIYWTSTRKSG
VPDRFSGSGSGTDFTLTISSLQAEDVAVYYCTQSFILRTFGQGTKVEIKRTVAAPSVFIFPPSDE
QLKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID NO. 10
DIVMTQSPLSLPVTPGEPASISCRSSQPLVHSNRNTYLHWYQQKPGQAPRLLIYKVSNRFSGV
PDRFSGSGSGTDFTLKISRVEAEDVGVYYCGQGTQVPYTFGQGTKLEIKRTVAAPSVFIFPPSD
EQLKSGTASVVCLLNNFYPREAKVQWKVDNALQSGNSQESVTEQDSKDSTYSLSSTLTLSKAD
YEKHKVYACEVTHQGLSSPVTKSFNRGEC
SEQ ID NO. 11
SYVLTQPPSVSVAPGQTARITCGGNNIGSKSVHWYQQKPGQAPVLVVYDDNDRPSGLPERFS
GSNSGNTATLTISRVEAGDEADYYCQVWDSSSDHVVFGGGTKLTVLGQPKAAPSVTLFPPSSE
ELQANKATLVCLISDFYPGAVTVAWKADSSPVKAGVETTTPSKQSNNKYAASSYLSLTPEQWK
SHRSYSCQVTHEGSTVEKTVAPTECS
SEQ ID NO. 12
DIQMTQSPSSLSASVGDRVTITCRASQSISSYLNWYQQKPGKAPKLLIYAASSLQSGVPSRFSG
SGSGTDFTLTISSLQPEDFATYYCQQSYSTPPTFGQGTKVEIKRTVAAPSVFIFPPSDEQLKSGT
ASVVCLLNNFYPREAKVQWKVDNALQSGNSQESVTEQDSKDSTYSLSSTLTLSKADYEKHKVY
ACEVTHQGLSSPVTKSFNRGEC

1. A method for producing a heterodimeric antibody that comprises two distinct IgG CH3 domains that are capable of forming a CH3-CH3 interface, said method comprising the steps of:
providing:
(a) a first antibody or half-antibody comprising a first CH3 domain which comprises a positively charged amino acid residue at position 351 and 366, and
(b) a second antibody or half-antibody comprising a second CH3 domain which comprises a negatively charged amino acid residue at position 351 and 368,
wherein the numbering is according to EU numbering,
incubating the antibodies and/or half-antibodies of (a) and (b) together under reducing conditions to provide a reduced first antibody or half-antibody and a reduced second antibody or half-antibody; and
reoxidising the reduced antibodies and/or half-antibodies to obtain the heterodimeric antibody, wherein the IgG CH3 domains of the heterodimeric antibody do not comprise an arginine at position 409 wherein the heterodimeric antibody comprises an IgG Fc region, and wherein the IgG Fc region comprises the IgG1 or IgG2 core hinge region CPPC.

2. The method of claim 1, wherein the IgG CH3 domain is an IgG1 or an IgG2 CH3 domain, optionally wherein the CH3 domain is a human CH3 domain.

3. The method of any one of the preceding claims, wherein the first antibody or half-antibody and/or the second antibody or half-antibody is a human antibody or half-antibody.

4. The method of claim 3, wherein the antibody or half antibody is an IgG1 or an IgG2 antibody or half antibody.

5. The method of any one of the preceding claims, wherein the first antibody or half-antibody is an antibody having a first binding specificity and the second antibody or half-antibody is an antibody have a second distinct binding specificity.

6. The method of any one of the preceding claims, wherein the first antibody and/or the second antibody is a homodimeric antibody.

7. The method of any one of the preceding claims, wherein the first antibody and/or the second antibody is a heterodimeric antibody.

8. The method of claim 7, wherein the heterodimeric antibody is multivalent, optionally wherein the multivalent antibody is a bivalent, trivalent, tetravalent antibody or having up to six valences.

9. The method of claim 7 or 8, wherein the heterodimeric antibody is a multi-specific antibody, optionally wherein the multi-specific antibody is a bi-specific, tri-specific or quattro-specific antibody.

10. The method of any one of claims 7 to 9, wherein the heterodimeric antibody comprises two non-identical light chains.

11. The method of any one of the preceding claims, wherein the first CH3 domain comprises: 351K and 366R, 351R and 366K, 351K and 366K, or 351R and 366R.

12. The method of any one of the preceding claims, wherein the second CH3 domain comprises: 351D and 368E, 351E and 368D, 351D and 368D, or 351E and 368E.

13. The method of any one of the preceding claims, wherein the first CH3 domain comprises 351K and 366K and the second CH3 domain comprises 351D and 368E.

14. The method of any one of the preceding claims, wherein the first and/or second antibody or half-antibody of a) and b) are Fc engineered.

15. The method of any one of the preceding claims, wherein the first and/or second antibody or half-antibody of a) and b) are Fc silenced or Fc enhanced.

16. The method of any one of the preceding claims, wherein the first and/or second antibody or half-antibody of a) and b) comprise a CH2 domain having an ADCC-affecting mutation at position 235 and/or 236.

17. The method of any one of the preceding claims, wherein the second antibody or half-antibody is obtained independently from the first antibody or half-antibody.

18. The method of any one of the preceding claims, wherein the reducing conditions comprise:
(a) incubating the antibodies or half-antibodies in the presence of a reducing agent, optionally wherein the reducing agent comprises or is selected from the group consisting of: 2-mercaptoethylamine (2-MEA), dithiothreitol (DTT), dithioerythritol (DTE), glutathione, tris(2-carboxyethyl)phosphine (TCEP), L-cysteine, beta-mercapto-ethanol, thioglycolate, cysteamine, homocysteine, penicillamine, and sodium borohydride; and/or
(b) incubating the antibodies or half-antibodies at a pH of between 6.0 and 12.0; and/or
(c) incubating the antibodies or half-antibodies at a redox potential of between −150 and −600 mV, optionally wherein the redox potential is between −250 and −400 mV.

19. The method of any one of the preceding claims, wherein the method further comprises the step of enriching for and/or isolating the heterodimeric antibody obtained after reoxidisation.

20. The method of claim 19, wherein the heterodimeric antibody is enriched for and/or isolated using a method comprising or selected from the group consisting of: precipitation, centrifugation, filtration, size-exclusion chromatography, affinity chromatography, cation- and/or anion-exchange chromatography, and hydrophobic interaction chromatography.

21. An isolated heterodimeric antibody obtainable by the methods according to any one of the preceding claims.

22. An isolated heterodimeric antibody comprising a first IgG CH3 domain and a second IgG CH3 domain, wherein the first CH3 domain and the second CH3 domain are capable of forming a CH3-CH3 interface,
wherein the first CH3 domain comprises one or more of the amino acid variants 366K, 366R, 351K or 351R, and
wherein the second CH3 domain comprises one or more of the amino acid variants 351E, 351D, 368E or 368D,
said heterodimeric antibody further comprising two light chains that have non-identical sequences, wherein the IgG CH3 domains of the heterodimeric antibody do not comprise an arginine at position 409, wherein the heterodimeric antibody comprises and IgG Fc region, and wherein the IgG Fc region comprises the IgG1 or IgG2 core hinge region CPPC.

23. The isolated heterodimeric antibody of claim 22, wherein the first CH3 domain comprises 351K and 366K and the second CH3 domain comprises 351D and 368E.

24. A pharmaceutical composition comprising an isolated heterodimeric antibody according to any one of claims 21 to 23, and a pharmaceutically acceptable carrier.

25. The method of any one of the claims 1-20, wherein the first antibody or half-antibody and/or the second antibody or half-antibody comprises an antibody binding domain, a scFv, a ligand, protein receptor or cytokine.

26. The method of claim 25, wherein the heterodimeric antibody is a bi- or multifunctional fusion protein.


SEQUENCE LISTING (12 sequences)

SEQ ID NO: 1 - protein, 449 aa, synthetic construct
  REGION 1..449: note = HC sequence
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 452 aa, synthetic construct
  REGION 1..452: note = HC sequence
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 451 aa, synthetic construct
  REGION 1..451: note = HC sequence
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 453 aa, synthetic construct
  REGION 1..453: note = HC sequence
EVQLVQSGAEVKKPGESLKISCKGSGYSFTSYWIGWVRQMPGKGLEWMGIIFPDDSDTRYSPSFQGQVTISADKSISTAYLQWSSLKPSDTAMYYCVRLGGYSGYAEDFVDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 449 aa, synthetic construct
  REGION 1..449: note = HC sequence
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 6 - protein, 452 aa, synthetic construct
  REGION 1..452: note = HC sequence
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 7 - protein, 451 aa, synthetic construct
  REGION 1..451: note = HC sequence
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 8 - protein, 453 aa, synthetic construct
  REGION 1..453: note = HC sequence
EVQLVQSGAEVKKPGESLKISCKGSGYSFTSYWIGWVRQMPGKGLEWMGIIFPDDSDTRYSPSFQGQVTISADKSISTAYLQWSSLKPSDTAMYYCVRLGGYSGYAEDFVDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 9 - protein, 219 aa, synthetic construct
  REGION 1..219: note = LC sequence
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 219 aa, synthetic construct
  REGION 1..219: note = LC sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 214 aa, synthetic construct
  REGION 1..214: note = LC sequence
SYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 214 aa, synthetic construct
  REGION 1..214: note = LC sequence
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC